(19) United States
(12) Patent Application Publication
(10) Pub. No.: US 2026/0339104 A1
(43) Pub. Date: Oct. 29, 2026

(54) Electronic Device And Operation Method Thereof

(71) Applicant: Semiconductor Energy Laboratory Co., Ltd., Kanagawa-ken (JP)

(72) Inventors: Ryo HATSUMI, Hadano (JP); Hisao IKEDA, Zama (JP); Daiki NAKAMURA, Atsugi (JP); Tomotaka NISHIMURA, Kawasaki (JP)

(73) Assignee: Semiconductor Energy Laboratory Co., Ltd., Kanagawa-ken (JP)

(21) Appl. No.: 19/653,588

(22) Filed: Apr. 21, 2026

(30) Foreign Application Priority Data
Apr. 25, 2025 (JP) 2025-073361

(51) Int. Cl. G02B 27/01; A61B 3/00; A61B 3/12

(52) U.S. Cl. CPC G02B 27/0172; A61B 3/005; A61B 3/12; G02B 2027/0114; G02B 2027/0138; G02B 2027/0156; G02B 2027/0178

(57) ABSTRACT

An electronic device capable of fundus image capturing is provided. The electronic device is a goggles-type device having a fundus image capturing function, and a light-emitting element and a light-receiving element are provided in a pixel of a display panel. In a display-viewing mode, visible light emitted from the pixel forms an image on a retina through a first optical system including a half mirror that has different functions depending on whether the light is visible light or infrared light. At this time, in the first optical system, an optical path that reduces a focal length is formed with reflective refraction. In a fundus image capturing mode, infrared light emitted from the pixel is used to form an optical path in which a reflection path is omitted. Thus, loss of light due to the half mirror can be minimized, and an image with a high S/N ratio can be obtained.

### BACKGROUND OF THE INVENTION

### 1. Field of the Invention

One embodiment of the present invention relates to an electronic device and an operation method thereof.

Note that one embodiment of the present invention is not limited to the above technical field. The technical field of one embodiment of the invention disclosed in this specification and the like relates to an object, a method, or a manufacturing method. One embodiment of the present invention relates to a process, a machine, manufacture, or a composition of matter. Specifically, examples of the technical field of one embodiment of the present invention disclosed in this specification include a semiconductor device, a display device, a liquid crystal display device, a light-emitting apparatus, a lighting device, a power storage device, a memory device, an image capturing device, a method for operating any of them, and a method for manufacturing any of them.

In this specification and the like, a semiconductor device generally means a device that can function by utilizing semiconductor characteristics. A transistor and a semiconductor circuit are embodiments of semiconductor devices. In some cases, a memory device, a display device, an image capturing device, or an electronic device includes a semiconductor device.

### 2. Description of the Related Art

Goggles-type devices and glasses-type devices have been developed as electronic devices for extended reality (XR). Note that XR is a general term for virtual reality (VR), augmented reality (AR), mixed reality (MR), and the like.

Typical examples of display panels that can be used for these electronic devices include a display device including a liquid crystal element and a display device including an organic electroluminescent (EL) element, a light-emitting diode (LED), or the like.

A display device including an organic EL element does not need a backlight, which is necessary for a liquid crystal display device, and thus can have advantages such as thinness, lightweight, high contrast, and low power consumption. Patent Document 1, for example, discloses examples of an electronic device for VR using an organic EL element and an optical device used for the electronic device. Patent Document 2 discloses an example of a display device including a light-receiving element in a pixel.

### References

### Patent Document 1

PCT International Publication No. 2023/126740

### Patent Document 2

PCT International Publication No. 2023/052892

### SUMMARY OF THE INVENTION

A smartwatch, which is a watch-type portable information terminal, is known as a typical example of wearable devices for health care. Many available smartwatches are capable of measuring physiological data such as heart rate, blood pressure, temperature, and blood oxygen level. Meanwhile, glasses-type or goggles-type electronic devices, which are other wearable devices, do not typically have the function of obtaining user's physiological data, while being in use for health care, such as remote treatment, rehabilitation, and learning.

Fundus examination in ophthalmology is essential for early detection of abnormalities in a retina, a macula portion, and the like. A fundus is the only portion in a human body where a blood vessel can be directly observed. The finding of fundus conditions is useful for predicting the conditions such as hypertension and diabetes, so that the presence or progression of diseases can be checked.

However, fundus examination has been available only in medical examination or the like in an ophthalmological institution. A fundus camera for medical facilities is composed of a plurality of optical systems and relatively large, which cannot be owned by individuals. Even a fundus camera with a relatively small size has required a person who capture an image separately from the user, and it has been difficult for the user to capture a fundus image by themselves. From these problems, a fundus camera that is small and enables image capturing by the user themselves has been desired.

In view of the above, an object of one embodiment of the present invention is to provide an electronic device having a function of fundus image capturing. Another object is to provide an electronic device that enables fundus image capturing by a user themselves. Another object is to provide an electronic device having functions of a goggles-type device and a fundus camera. Another object is to provide an electronic device that detects operation data on a user's eye or the vicinity thereof. Another object is to provide a novel electronic device. Another object is to provide a novel semiconductor device or the like. Another object is to provide an operation method of the electronic device.

Note that the description of these objects does not preclude the existence of other objects. One embodiment of the present invention does not need to achieve all of these objects. Other objects will be apparent from and can be derived from the description of the specification, the drawings, the claims, and the like.

One embodiment of the present invention relates to an electronic device capable of capturing an image of the fundus of an eye.

One embodiment of the present invention is an electronic device including a display panel, a first optical system, a second optical system, and a position adjustment mechanism. The display panel includes a pixel including a first light-emitting element, a second light-emitting element, and a light-receiving element. The first light-emitting element has a function of emitting visible light. The second light-emitting element has a function of emitting infrared light. The light-receiving element has a function of performing photoelectric conversion of the infrared light. The first optical system faces a display surface of the display panel. The first optical system includes a half mirror and a reflective polarizing plate. The half mirror has a function of partially transmitting transmit and partially reflecting the visible light and to transmit the infrared light. The reflective polarizing plate has a function of transmitting or reflecting the visible light in accordance with a polarization state of the visible light and to transmit the infrared light regardless of a polarization state of the infrared light. The position adjustment mechanism has a function of moving the second optical system to a first position or a second position. The visible light emitted from the pixel forms an image on an object through the first optical system when the second optical system is at the first position. The infrared light emitted from the pixel forms an image on the object through the second optical system and the first optical system and infrared light reflected by the object forms an image on the pixel through the first optical system and the second optical system when the second optical system is at the second position.

In the first optical system, a linear polarizing plate, a first retardation plate, the half mirror, a second retardation plate, and the reflective polarizing plate can be arranged in one direction in this order from a display panel side.

The second optical system can have a positive power.

The first position can be out of the optical path of light that is emitted from the pixel and reaches the first optical system, and the second position can be in the optical path of the light that is emitted from the pixel and reaches the first optical system.

The first light-emitting element and the second light-emitting element can be organic EL elements, and the light-receiving element can be a photoelectric conversion element including an organic layer for a photoelectric conversion layer.

The position adjustment mechanism can include a stepper motor.

The pixel preferably includes a transistor connected to the first light-emitting element, the second light-emitting element, and the light-receiving element, and the transistor preferably includes a metal oxide in a channel formation region. The metal oxide preferably contains indium and oxygen.

Another embodiment of the present invention is a method of operating an electronic device worn in front of an eye. The electronic device including a first optical unit corresponding to one of left and right eyes and a second optical unit corresponding to the other of the left and right eyes. The method includes switching a first mode and a second mode. In the first mode, visible light is emitted from a pixel of a display panel included in each of the first optical unit and the second optical unit, and the visible light forms an image on an object through a first optical system included in each of the first optical unit and the second optical unit. In the second mode, in one of the first optical unit and the second optical unit, display with the visible light is performed on the display panel to adjust the thickness of a crystalline lens of each of the eyes, and in the other of the first optical unit and the second optical unit, a second optical system is inserted between the display panel and the first optical system, infrared light is emitted from the pixel of the display panel, the infrared light forms an image on the object through the second optical system and the first optical system, and infrared light reflected by the object forms an image on the pixel through the first optical system and the second optical system.

According to one embodiment of the present invention, an electronic device having a function of fundus image capturing can be provided. Alternatively, an electronic device that enables fundus image capturing by a user themselves can be provided. Alternatively, an electronic device having functions of a goggles-type device and a fundus camera can be provided. Alternatively, an electronic device that detects operation data on a user's eye or the vicinity thereof can be provided. Alternatively, a novel electronic device can be provided. Alternatively, a novel semiconductor device or the like can be provided. Alternatively, an operation method of the electronic device can be provided.

Note that the description of these effects does not preclude the existence of other effects. One embodiment of the present invention does not necessarily have all of these effects. Other effects can be derived from the description of the specification, the drawings, and the claims.

### BRIEF DESCRIPTION OF THE DRAWINGS

FIG. 1 illustrates an electronic device.

FIGS. 2A and 2B illustrate an electronic device.

FIGS. 3A and 3B illustrate an electronic device.

FIG. 4 illustrates a fundus camera.

FIG. 5A illustrates light returning to a pixel, and FIG. 5B illustrates an angle of view at the time of fundus image capturing.

FIG. 6 illustrates an operation of an electronic device.

FIG. 7A illustrates an operation of an electronic device, and FIG. 7B illustrates the electronic device.

FIGS. 8A to 8C illustrate structure examples of pixels.

FIG. 9 illustrates a catadioptric optical system and an optical path of visible light.

FIG. 10 shows optical characteristics of a half mirror.

FIGS. 11A to 11G each illustrate a half mirror and a support.

FIG. 12 illustrates a catadioptric optical system and an optical path of infrared light.

FIGS. 13A and 13B illustrate a display panel.

FIGS. 14A and 14B each illustrate a pixel circuit.

FIGS. 15A and 15B each illustrate a pixel circuit.

FIG. 16 is a perspective view illustrating an electronic device.

FIG. 17 illustrates a structure example of a display device.

FIG. 18 illustrates a structure example of a display device.

FIG. 19 shows a structure example of a display device.

FIG. 20 shows a structure example of a display device.

FIG. 21 illustrates a structure example of a display device.

FIG. 22 illustrates a structure example of a display device.

FIGS. 23A and 23B illustrate a transistor.

### DETAILED DESCRIPTION OF THE INVENTION

Embodiments will be described in detail with reference to the drawings. Note that the present invention is not limited to the following description, and it will be readily appreciated by those skilled in the art that modes and details of the present invention can be modified in various ways without departing from the spirit and scope of the present invention. Accordingly, the present invention should not be construed as being limited to the description in the following embodiments. Note that in structures of the invention described below, the same portions or portions having similar functions are denoted by the same reference numerals in different drawings, and the description thereof is not repeated in some cases. The same components are denoted by different hatching patterns in different drawings, or the hatching patterns are omitted in some cases.

Even in the case where a single component is illustrated in a circuit diagram, the component may be composed of a plurality of parts as long as there is no functional inconvenience. For example, in some cases, a plurality of transistors that operate as a switch are connected in series or in parallel. In some cases, capacitors are divided and arranged in a plurality of positions.

One conductor has a plurality of functions such as a wiring, an electrode, and a terminal in some cases. In this specification, a plurality of names are used for the same component in some cases. Even in the case where components are illustrated in a circuit diagram as if they were directly connected to each other, the components may actually be connected to each other through one or more conductors. In this specification, even such a structure is included in direct connection.

The expression "connection" in this specification includes "electrical connection", for example. Note that the expression "electrical connection" is used in some cases to specify the connection relation of a circuit element as an object. The term "electrical connection" includes "direct connection" and "indirect connection". The expression "A and B are directly connected" means that A and B are connected to each other without a circuit element such as a transistor or a switch (note that a wiring is not a circuit element) therebetween. By contrast, the expression "A and B are indirectly connected" means that A and B are connected to each other with at least one circuit element therebetween.

For example, assuming that a circuit including A and B is in operation, the circuit can be specified as "A and B are indirectly connected" as an object when electric signal transmission and reception or electric potential interaction between A and B occurs at some point during the operation period of the circuit. Note that even when neither electric signal transmission and reception nor electric potential interaction between A and B occurs at some point during the operation period of the circuit, the circuit can be specified as "A and B are indirectly connected" as long as electric signal transmission and reception or electric potential interaction between A and B occurs at another point during the operation period of the circuit.

Examples of the case where the expression "A and B are indirectly connected" can be used include the case where A and B are connected to each other through a source and a drain of at least one transistor. By contrast, examples of the case where the expression "A and B are indirectly connected" cannot be used include the case where an insulator is present on the path from A to B. Specific examples thereof include the case where a capacitor is connected between A and B and the case where a gate insulating film of a transistor or the like is present between A and B. In such cases, the expression "a gate (A) of a transistor and a source or a drain (B) of the transistor are indirectly connected" cannot be used.

Another example of the case where the expression "A and B are indirectly connected" cannot be used is the case where a plurality of transistors are connected through their sources and drains on the path from A to B and a constant electric potential V is supplied from a power source, GND, or the like to a node between one of the transistors and another one of the transistors.

### Embodiment 1

In this embodiment, an electronic device and a an operation method thereof according to one embodiment of the present invention will be described.

One embodiment of the present invention is an electronic device that is worn in front of eyes, can be used as a goggles-type device compatible with XR, and has a fundus image capturing function. Goggles-type devices have wider application in daily life and are suitable as health care devices. The electronic device is useful for daily health management because a fundus reflects physiological abnormalities. The goggles-type device is assumed to be used by an individual so that a user themselves can capture a fundus image.

The goggles-type device of one embodiment of the present invention includes a display panel, a first optical system, and a second optical system. The display panel includes a first light-emitting element functioning as a display element, a second light-emitting element functioning as an observation light source, and a light-receiving element for fundus image capturing. Note that each of the light-emitting elements can also be referred to as a light-emitting device. A light-receiving element can also be referred to as a light-receiving device.

The first light-emitting element, the second light-emitting element, and the light-receiving element are provided in a pixel of the display panel. A display-viewing mode (hereinafter referred to as a display mode) uses visible light emitted from the first light-emitting element. Meanwhile, a fundus image capturing mode (hereinafter referred to as an imaging mode) uses infrared light emitted from the second optical system.

A half mirror and a reflective polarizing plate included in the first optical system each exhibit different functions depending on whether the light is visible light or infrared light. Specifically, the half mirror has functions of partially transmitting and partially reflecting visible light and transmitting infrared light. The reflective polarizing plate has a function of reflecting or transmitting visible light in accordance with the polarization state of the visible light, and has a function of transmitting infrared light regardless of the polarization state of the infrared light.

With this structure, in the display mode, visible light emitted from the first light-emitting element forms an image on a retina through the first optical system. At this time, an optical path that reduces a focal length by including a reflection path is formed by the functions of the half mirror and the reflective polarizing plate in the first optical system. This makes it possible to downsize the optical system.

In the imaging mode, the retina is irradiated with infrared light emitted from the second light-emitting element through the first optical system. The half mirror transmits infrared light as described above, so that light loss due to the half mirror can be minimized. In addition, the reflective polarizing plate can transmit infrared light regardless of the polarization state. Accordingly, the amount of light reaching the fundus can be increased, and the amount of light that returns from the fundus to the pixel can also be increased.

However, in the first optical system, the focus is set along the optical path with the reflection path in the display mode. Thus, in the case where an optical path that passes through the half mirror and the reflective polarizing plate is formed, light reflected from the fundus cannot form an image on the pixel. Hence, an optical path length is corrected by, for example, inserting the second optical system between the display panel and the first optical system, to allow focus adjustment to be performed such that reflected light forms an image on the pixel.

The focus adjustment is affected by the thickness of the crystalline lens of a human eye. Humans naturally change the thickness of their crystalline lens to focus light emitted by a viewed object on their retina. Thus, in the focus adjustment, some kind of display performed on the display panel needs to be viewed by a user.

The functions of the left and right eyes are linked; even when, with light entering one eye being blocked, the other eye views some kind of image and performs focusing (adjusting the thickness of the crystalline lens), the one eye performs focusing in response to the other eye. With the utilization of such properties, one of two optical units (each including the display panel and the first optical system) corresponding to the left and right eyes is operated in the display mode, where display with visible light is viewed and the focus adjustment of both eyes is performed. Furthermore, the other of the optical units is operated in the imaging mode, where the optical path length is corrected and infrared light is used to perform an imaging operation. Consequently, a fundus image can be captured while the pixel is in focus.

The amount of light extracted to the outside after being reflected by the fundus is small. That is, the signal is weak, and therefore the S/N ratio of data that can be obtained by the light-receiving element is likely to be low. In one embodiment of the present invention, the light utilization efficiency can be increased as described above and accordingly image capturing with less noise and a high S/N ratio can be achieved.

As the first optical system, for example, a catadioptric optical system can be used. A catadioptric optical system has a structure in which a plurality of components (optical components) are combined. A mechanism in which such a structure is included in a housing is simply called a lens. It is also called a pancake lens in some cases because of its thin shape.

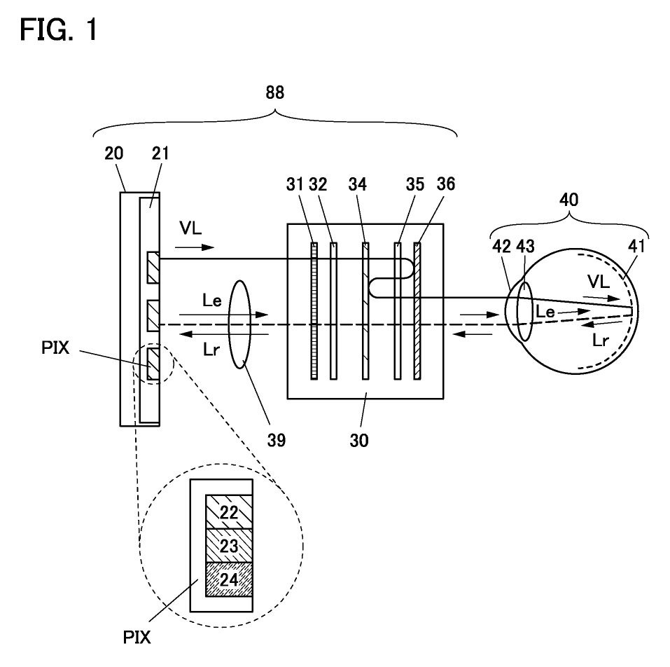

FIG. 1 is a conceptual diagram illustrating an optical path between an eyeball and a component included in the electronic device of one embodiment of the present invention, and part of the diagram is enlarged. An electronic device 88 has a function of an electronic device used for XR application and includes a display panel 20, a catadioptric optical system 30, and a focus adjustment optical system 39. In FIG. 1, cross sections of these components are simplified. Note that in FIG. 1, only the main components of the catadioptric optical system 30 are illustrated, and details thereof will be described later.

The display panel 20 is placed so that a display surface is perpendicular to an optical axis of the catadioptric optical system 30. In other words, the catadioptric optical system 30 is placed to face the display surface of the display panel 20. Note that the term "perpendicular" in this specification and the like indicates that the angle formed between two straight lines is greater than or equal to 85° and less than or equal to 95°. Here, one of the two straight lines is the optical axis of the catadioptric optical system 30, and the other is a straight line parallel to the display surface.

The user can see an image displayed on the display panel 20 by bringing their eye 40 closer to the vicinity of the catadioptric optical system 30. The user views the image with a viewing angle widened by the catadioptric optical system 30, and thus can feel a sense of immersion and a realistic sensation. Furthermore, by the image capturing operation of the electronic device, a fundus image can be obtained and their health conditions can be checked.

The display panel 20 includes a pixel PIX in a display portion 21. The pixel PIX includes a subpixel 22 including a light-emitting element that emits visible light, a subpixel 23 including a light-emitting element that emits infrared light, and a subpixel 24 including a light-receiving element that performs photoelectric conversion of infrared light. The subpixel 22 can be used as a light source for display, and the subpixel 23 can be used as a light source for image capturing. The subpixel 24 can be used for fundus image capturing. Although the details will be described later, the number of subpixels 22 included in one pixel PIX is not limited to one and may be two or more. In addition, either the subpixel 23 or the subpixel 24 is not provided in some cases.

The catadioptric optical system 30 includes a linear polarizing plate 31, a retardation plate 32, a half mirror 34, a retardation plate 35, and a reflective polarizing plate 36. The half mirror 34 has functions of partially transmitting and partially reflecting visible light and transmitting infrared light. The reflective polarizing plate 36 has a function of reflecting or transmitting visible light in accordance with the polarization state of the visible light, and has a function of transmitting infrared light regardless of the polarization state of the infrared light. Note that FIG. 1 does not illustrate the components changing the traveling direction of light, such as a concave surface and a lens. Details of these components are described later.

In the structure illustrated in FIG. 1, the eye 40 is focused on the display surface of the display panel 20. At this time, visible light VL emitted from the subpixel 22 is repeatedly reflected and refracted while changing the polarization state in the catadioptric optical system 30, thereby forming an image on a retina 41 through a cornea 42 and a crystalline lens 43 functioning as lenses. In other words, the user can view the image on the display panel 20.

Note that in this specification, visible light refers to, for example, light with a wavelength of 360 nm to 830 nm or light emitted from the display panel within the wavelength range of blue to red (e.g., 450 nm to 780 nm). In general, infrared light refers to an electromagnetic wave with a wavelength longer than that of the above-described visible light and shorter than that of a microwave. In one embodiment of the present invention, however, near-infrared light (e.g., light with a wavelength of 780 nm to 2 μm) with relatively high energy is preferably used, in which case the photoelectric conversion is facilitated. The wavelength of infrared light used can be selected in consideration of the characteristics of the light-emitting element, the light-receiving element, the half mirror 34, and the reflective polarizing plate 36.

Here, since the pixel PIX and the retina 41 are conjugated, the visible light VL reflected by the retina 41 forms an image on the pixel PIX through the crystalline lens 43, the cornea 42, and the catadioptric optical system 30. This means that the catadioptric optical system 30 alone enables fundus image capturing using visible light reflected by the fundus. However, the normal optical path of the catadioptric optical system 30 includes a transmission path and a reflection path that are related to the half mirror 34; accordingly, light loss is large and it is difficult to sufficiently increase the light utilization efficiency. That is, it is difficult to increase the S/N ratio of an image that can be captured.

In view of the above, in one embodiment of the present invention, infrared light emitted from the subpixel 23 is used for the image capturing operation, and the half mirror 34 and the reflective polarizing plate 36 that transmit infrared light are used. Accordingly, light loss due to the half mirror 34 can be minimized. Furthermore, infrared light can reach the fundus while maintaining high transmittance of the whole optical system because it can pass through the reflective polarizing plate regardless of the polarization state.

The focus is set along the optical path with the reflection path in the catadioptric optical system 30. Thus, owing to the half mirror 34 and the reflective polarizing plate 36 having the above characteristics, infrared light (emission light Le) that is illumination light emitted from the pixel PIX cannot form an image on the fundus. In addition, infrared light (reflected light Lr) reflected from the fundus cannot form an image on the pixel PIX. Hence, for example, the focus adjustment optical system 39 is inserted between the display panel and the first optical system to correct an optical path length, and focus adjustment is performed such that the reflected light Lr forms an image on the pixel PIX.

The focus adjustment optical system 39 is used to adjust the focal length of the catadioptric optical system 30 that shifts due to the use of infrared light; however, without being limited thereto, another method for adjusting a focal point described later can also be used. This embodiment mainly explains an example in which the focus adjustment optical system 39 is used.

The optical path of the visible light VL in the catadioptric optical system 30 includes a transmission path and a reflection path that are related to the half mirror 34. Therefore, when the transmittance and reflectance of the half mirror 34 are each 50 %, the loss of the emitted light due to the half mirror 34 reaches 75 %. Furthermore, if the same optical path is used for image capturing, the loss of the reflected light also reaches 75 %.

By contrast, infrared light (the emission light Le and the reflected light Lr) passes through the half mirror 34 in the catadioptric optical system 30, and therefore when the transmittance of the half mirror 34 is ideally 100 %, there is no light loss due to the half mirror 34. In other words, the light utilization efficiency can be increased, and an image with a higher S/N ratio can be captured.

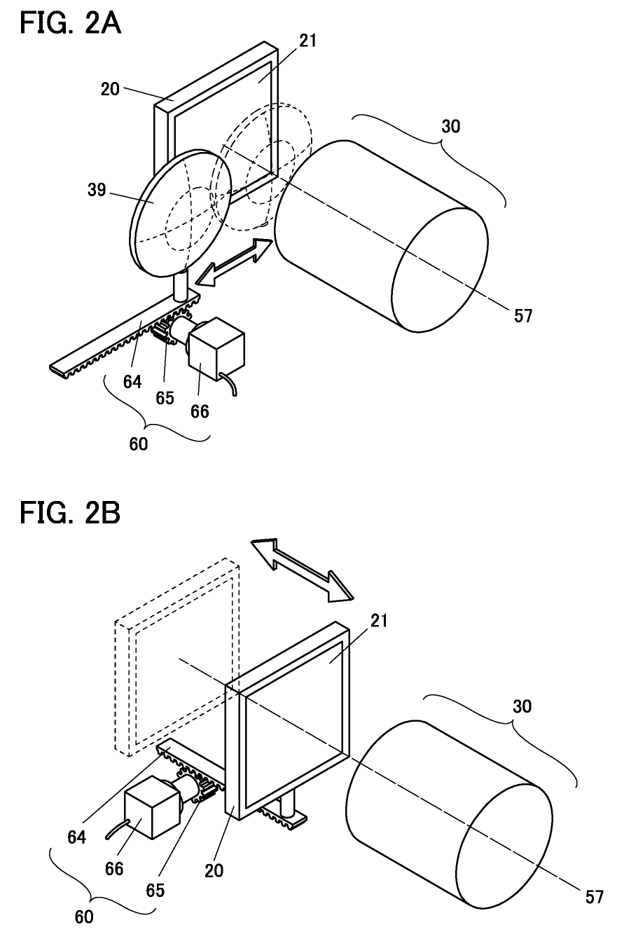

FIG. 1 is a conceptual diagram illustrating a mode in which the focus adjustment optical system 39 is inserted only in the optical path of infrared light (the emission light Le and the reflected light Lr); however, the actual focus adjustment optical system 39 is not fixed at a predetermined position. As illustrated in a perspective view of FIG. 2A, the focus adjustment optical system 39 is preferably movable so as to be inserted in an optical path of light emitted from the display panel 20.

For example, the focus adjustment optical system 39 is configured to be capable of moving between a first position out of the optical path of light that is emitted from the display panel 20 and reaches the catadioptric optical system 30 and a second position in the optical path, as illustrated in FIG. 2A. The second position is preferably a position such that the optical axis of the focus adjustment optical system 39 overlaps with the extension line of an optical axis 57 of the catadioptric optical system 30.

The focus adjustment optical system 39 is preferably connected to a position adjustment mechanism. In the example shown in FIG. 2A, the focus adjustment optical system 39 can be slid between the first and second positions by a position adjustment mechanism 60, which combines a rack 64 connected to the focus adjustment optical system 39 and a pinion 65 connected to a power mechanism 66.

The above is an example and one embodiment of the present invention is not limited thereto. For example, a ball screw mechanism or a structure using a screw gear can also be used. A stepper motor is preferably used as the power mechanism 66. The stepper motor can control the rotation angle using pulse signals and enables precise position adjustment.

Although an example in which the focus adjustment optical system 39 is used to adjust the focal length of the catadioptric optical system 30 that shifts due to the use of infrared light is described above, one embodiment of the present invention is not limited thereto.

For example, as illustrated in FIG. 2B, the distance between the catadioptric optical system 30 and the display panel 20 is increased so that the focal length is adjusted. This structure eliminates the need for the focus adjustment optical system 39 and reduces the number of components. Connecting the position adjustment mechanism 60 to the display panel 20 or the catadioptric optical system 30, as illustrated in FIG. 2B, is effective in obtaining such a structure. Note that the structure illustrated in FIG. 2B and the structure using the focus adjustment optical system 39 can be combined.

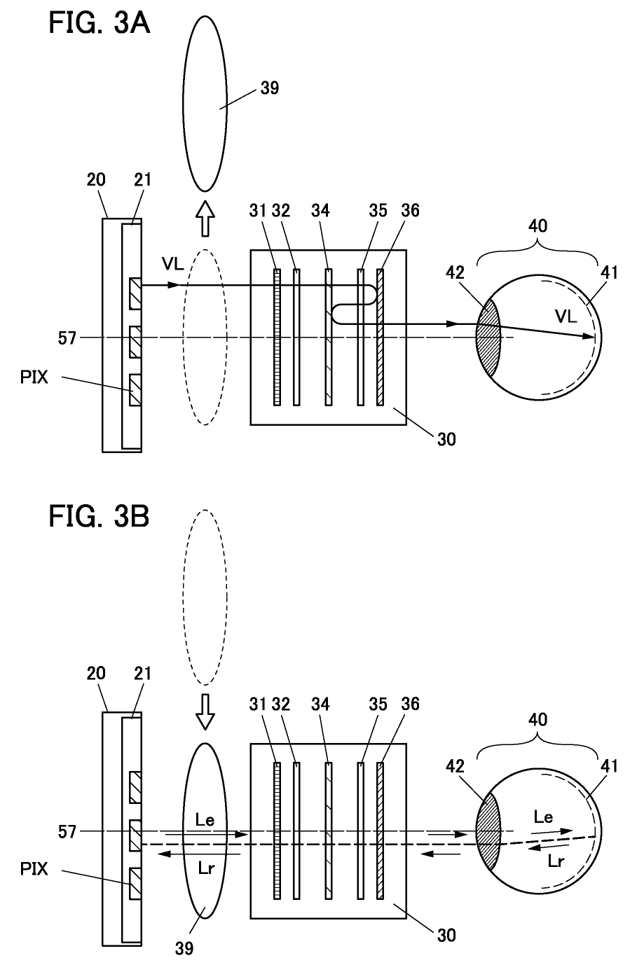

Next, the optical paths of the catadioptric optical system 30 are briefly described with reference to FIGS. 3A and 3B. FIG. 3A illustrates a normal optical path of the catadioptric optical system 30 using the visible light VL. The normal optical path can also be referred to as an optical path in the display mode. In the display mode, the focus adjustment optical system 39 is positioned out of the optical path.

As described above, the half mirror 34 has functions of partially transmitting and partially reflecting visible light and transmitting infrared light. The reflective polarizing plate 36 has a function of reflecting or transmitting visible light in accordance with the polarization state of the visible light, and has a function of transmitting infrared light regardless of the polarization state of the infrared light.

In the structure of the catadioptric optical system 30 illustrated in FIG. 3A, each of a combination of the linear polarizing plate 31 and the retardation plate 32 and a combination of the retardation plate 35 and the reflective polarizing plate 36 functions as a circularly polarizing plate. Therefore, light that passes through the half mirror 34 to be incident on the retardation plate 35 is circularly polarized light, and the retardation plate 35 converts the light into linearly polarized light whose polarization plane is aligned with the reflection axis of the reflective polarizing plate 36.

The linearly polarized light reflected by the reflective polarizing plate 36 is incident on the retardation plate 35 and converted into circularly polarized light, and the light is reflected by the half mirror 34, so that the handedness of the circularly polarized light is reversed. The circularly polarized light with the reversed handedness is incident on the retardation plate 35 and then converted into linearly polarized light whose polarization plane is orthogonal to the reflection axis of the reflective polarizing plate 36. That is, the linearly polarized light has the same oscillation direction as the transmission axis of the reflective polarizing plate 36, and therefore passes through the reflective polarizing plate 36. Thus, in the case where the visible light VL is used, the normal optical path of the catadioptric optical system 30 can be formed.

FIG. 3B illustrates the optical path of the catadioptric optical system 30 using infrared light (the emission light Le and the reflected light Lr). The normal optical path can also be referred to as an optical path in the imaging mode. In the imaging mode, the focus adjustment optical system 39 is positioned on the optical path.

Here, as in the above description of optical path of the visible light VL, the focus of the catadioptric optical system 30 is set along the optical path with the reflection path. Accordingly, in the case where infrared light is used and the reflection path is omitted, the emission light Le cannot form an image on the retina 41. Furthermore, the reflected light Lr cannot form an image on the pixel PIX. Considering these, the focus adjustment optical system 39 is inserted in the optical path between the display panel 20 and the catadioptric optical system 30. This enables the emitted light Le to form an image on the retina 41 and the reflected light Lr to form an image on the pixel PIX.

Here, with the use of the focus adjustment optical system 39, the focal length extended by the omission of the reflection path is adjusted and reduced. Thus, a lens having positive power can be used for the focus adjustment optical system 39. The lens can be typically a convex lens or can be a combination of lenses in order to correct a variety of aberrations.

The half mirror 34 transmits infrared light in the optical path of the emission light Le illustrated in FIG. 3B. The reflective polarizing plate 36 transmits infrared light regardless of the polarization state. Thus, the property of the half mirror 34 partially transmitting and partially reflecting the visible light VL is not used, and accordingly the light utilization efficiency can be increased. Furthermore, the reflected light Lr is infrared light and therefore can pass through the reflective polarizing plate 36 and the half mirror 34, which can increase the light utilization efficiency as in the case of the optical path of the emitted light Le.

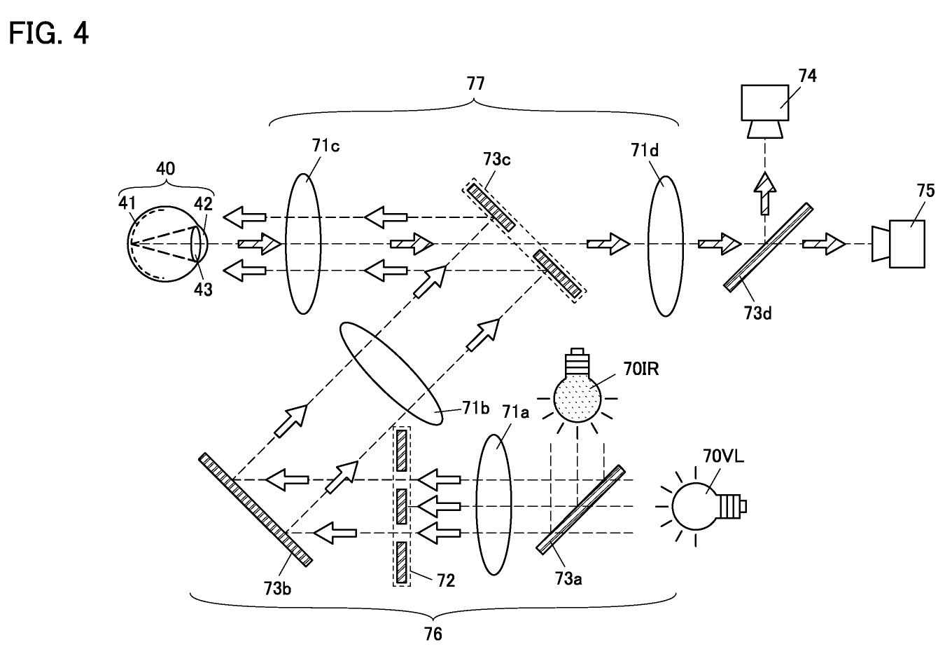

Next, an example of an optical system used in a conventional fundus camera is described with reference to FIG. 4. The conventional fundus camera has a structure in which two optical systems, an optical system 76 for lighting and an optical system 77 for observation, are combined.

A dichroic mirror 73a, a lens 71a, a ring slit 72, a reflective mirror 73b, a lens 71b, a perforated mirror 73c, and a lens 71c are provided in an optical path of the optical system 76. The lens 71c, the perforated mirror 73c, a lens 71d, and a dichroic mirror 73d are provided in the optical path of the optical system 77. Here, the lens 71c and the perforated mirror 73c are shared by the optical system 76 and the optical system 77. Here, an example in which the dichroic mirrors 73a and 73d have functions of transmitting visible light and reflecting infrared light is described.

The optical path of each optical system is described. Note that the lenses 71a to 71d have a function of appropriately refracting light such that the light comes into focus on a fundus including the retina 41 of the examinee's eye 40, the observation camera 74, or the imaging camera 75, which is the light's destination. The detailed descriptions of the lenses 71a to 71d are not detailed below.

In the optical system 76 for lighting, light emitted from an infrared light source 70IR or light emitted from a visible light source 70VL is emitted along the optical path through the dichroic mirror 73a. The light emitted along the optical path reaches the fundus including the retina 41 through the ring slit 72, the reflective mirror 73b, and the perforated mirror 73c. After passing through the ring slit 72, the light forms a donut shape and is reflected by a reflective surface on the outer side of a hole portion of the perforated mirror 73c, thereby reaching the eye 40.

In the optical system 77 for observation, part of the light reflected by the fundus is emitted toward the hole portion of the perforated mirror 73c. The light passing through the hole portion reaches the observation camera 74 or the imaging camera 75 through the dichroic mirror 73d.

The infrared light emitted from the infrared light source 70IR is used as observation light. The infrared light reflected after reaching the fundus passes through the hole portion of the perforated mirror 73c and is reflected by the dichroic mirror 73d to reach the observation camera 74. By confirming an image captured by the observation camera 74, an observer adjusts an imaging position and the focus of the imaging camera 75 to prepare for fundus image capturing. The infrared light used in observation has low luminosity and does not dazzle the examinee.

The visible light emitted from the visible light source 70VL is used as imaging light. The visible light that has been reflected after reaching the fundus partly passes through the perforated mirror 73c and the dichroic mirror 73d and reaches the imaging camera 75. Accordingly, a fundus image can be captured. With the use of the perforated mirror 73c and the like in this manner, light reflected by the cornea 42 and the crystalline lens 43 of the eye 40 is prevented from entering the optical system 77 so that noise can be reduced as much as possible.

For fundus image capturing, light needs to enter the eye through the pupil, be reflected by the fundus, and be extracted through the pupil. The amount of light reflected by the fundus that can be extracted through the pupil is said to be approximately 0.01 % to 0.1 % of the amount of incident light, and therefore improving the S/N ratio in fundus image capturing is difficult.

Thus, as the visible light source 70VL, an independent light source that can emit strong light such as a flashlight is used to increase the signal intensity. The conventional fundus camera tends to increase in device size because it requires a large number of components and optical paths of the two optical systems, for example.

In the electronic device of one embodiment of the present invention having a function of a goggles-type device as described above, the light (image) emitted from the pixel PIX of the display panel 20 forms an image on the fundus including the retina 41 of the eye 40 through the catadioptric optical system 30, as illustrated in FIG. 1. That is, the catadioptric optical system 30 can be regarded as having a first function as an optical system for visualizing display on the display panel 20.

The function of making the light emitted from the pixel PIX reach the fundus through the focus adjustment optical system 39 and the catadioptric optical system 30 is substantially the same as the function of making the light emitted from the visible light source 70VL for image capturing in a conventional fundus camera reach the fundus through the optical system 76 for lighting. In other words, the pixel PIX has a function of the visible light source 70VL for image capturing, and the focus adjustment optical system 39 and the catadioptric optical system 30 have a second function as the optical system 76 for lighting.

Furthermore, the function of making the light from the fundus form an image in the pixel PIX through the catadioptric optical system 30 and the focal adjustment optical system 39 is substantially the same as the function of making the light emitted from the fundus reach the imaging camera 75 through the optical system 77 for observation in the conventional fundus camera. In other words, the catadioptric optical system 30 and the focal adjustment optical system 39 each have a third function as an optical system 77 for observation in the conventional fundus camera.

Thus, the above-described first to third functions can be achieved by combining the display panel 20 including the light-emitting element and the light-receiving element in the pixel PIX, the catadioptric optical system 30, and the focus adjustment optical system 39 in one embodiment of the present invention. Furthermore, the observer, the observation light source, the observation camera, and the like can be unnecessary, and a fundus image can be taken by the user themselves. Consequently, a large-sized optical system for a fundus camera can be downsized, leading to an electronic device that can capture a fundus image with a size equal to that of a conventional goggles-type device.

Note that in the case where a light ray, a lens, and the like related to the functions of the catadioptric optical system 30 and the focus adjustment optical system 39 are completely ideal, light that has been emitted from the subpixel 23 including the light-emitting element and then reflected by the fundus returns to the subpixel 23. In other words, an image is not formed on the subpixel 24 that includes the light-receiving element and is adjacent to the subpixel 23.

However, actual image formation is affected by various lens aberrations and light diffraction. As a result, light is not focused to a point at an image forming position, resulting in a finite spot diameter. Lens aberrations can be reduced to some extent by selecting an appropriate shape and material, and by combining a plurality of lenses. Nevertheless, for an optical system of an electronic device that needs to be downsized at low cost, completely correcting aberrations is not practical.

Even if the aberrations can be completely corrected, light has the properties of waves and the lens always has a diffraction limit, inevitably resulting in a finite spot diameter. The aberrations and light diffraction may have greater adverse effects and the spot diameter may be easily increased because light goes back and forth between the pixel PIX and the fundus. In addition, the display panel 20 used in the goggles-type device has a pixel density of thousands of ppi and an extremely minute pixel size.

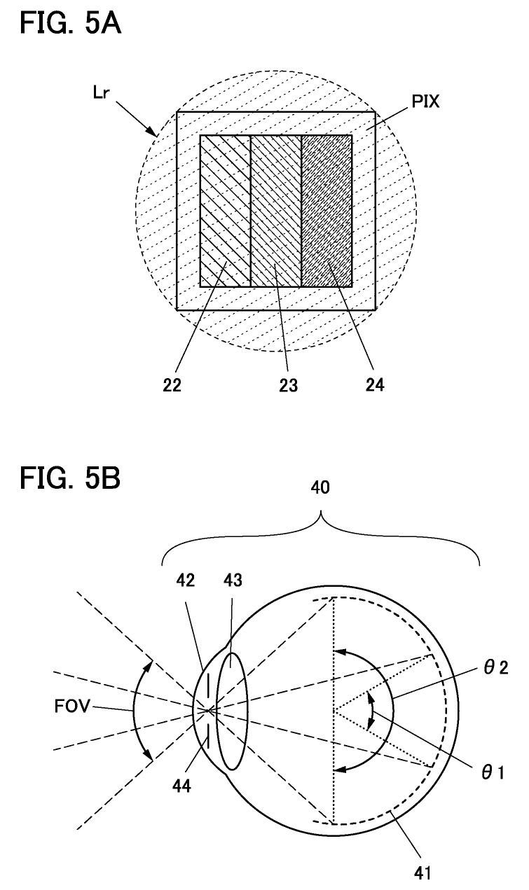

Owing to these complex factors, the subpixel 24 including the light-receiving element is within the spot diameter of the reflected light Lr that returns to the pixel PIX, as illustrated in the conceptual diagram in FIG. 5A. Thus, it can be said that the light emitted from the subpixel 23 and reflected by the fundus can enter the subpixel 24. That is, the light that is emitted from the pixel PIX and reflected by the fundus and returns to the pixel PIX can be used for fundus image capturing. In other words, an image of an object can be captured using the light that is emitted from the pixel PIX and reflected by the object and returns to the pixel PIX.

Note that the object is not limited to a human fundus, and can be an animal fundus, a portion corresponding to a fundus of an artificial object that imitates the eye, or the like. The shape, position, and size of the spot of the reflected light Lr illustrated in FIG. 5A are examples and may be changed depending on the modes of the pixel and the optical system.

As illustrated in FIG. 5B, the angle of view of the fundus camera depends on the angle of incidence on the pupil 44. The angle θ1 of view of a general fundus camera is approximately 45° to 60°. Therefore, a plurality of imaging operations with shifted positions are necessary to capture a wide range of the retina which occupies approximately 230° out of 360° of the inner wall of the eyeball.

Meanwhile, in the case where the optical system for the goggles-type device is used, the angle of incidence on the pupil 44 corresponds to a field of view (FOV) at the time of image viewing. Accordingly, when the catadioptric optical system for the goggles-type device is used for fundus image capturing, the angle of view θ2 can be larger than the angle of view θ1 and an image of a wide range of the retina can be captured at a time.

In the electronic device of one embodiment of the present invention, the focus on the fundus seen from the pixel side also needs to be adjusted at the time of image capturing, as in the case where a display is viewed by the eye.

A cornea and a crystalline lens, which are included in a human eye and each function as a lens, transmit light so that the light can form an image on the retina, whereby a visual object can be seen. The distance from an eye to a visual object is not uniform; therefore, to form an image of light emitted from the visual object on the retina, the refractive power of the eye for light needs to be changed by changing the thickness of the crystalline lens.

That is, focus adjustment by viewing the display panel needs to be performed in advance before image capturing. However, the illumination light (emission light Le) used for image capturing is infrared light with low luminosity, which is not suitable as the light (image) for the focus adjustment. Thus, in one embodiment of the present invention, visible light is used for a display operation for the focus adjustment by an optical unit corresponding to one of the left and right eyes, and infrared light is used for an image capturing operation by an optical unit corresponding to the other eye. Note that the term optical unit refers to a combination of the display panel 20 and the catadioptric optical system 30.

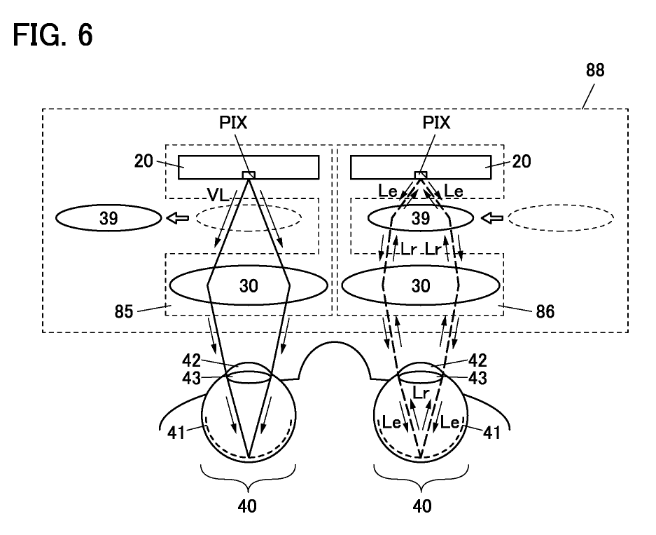

FIG. 6 illustrates an optical unit 85, an optical unit 86, and the focus adjustment optical systems 39 provided in the electronic device 88. The optical unit 85 and the optical unit 86 each correspond to one of the two eyes. The functions of the left and right eyes are linked; even when, with light entering one eye being blocked, the other eye views some kind of image and performs focusing (adjusting the thickness of the crystalline lens), the one eye performs focusing in response to the other eye.

On the basis of the aforementioned functions, the optical unit 85 corresponding to one of the left and right eyes is operated in the display mode (see FIG. 3A) using the normal optical path of the catadioptric optical system 30, and visualizes the display (visible light VL) so that the foci of both eyes are adjusted. Then, the other optical unit 86 is operated in the imaging mode (see FIG. 3B) in which the focus adjustment optical system 39 is inserted, and performs an imaging operation using the infrared light (emission light Le and reflected light Lr).

Thus, the infrared light (emission light Le) emitted from the pixel PIX in the optical unit 86 forms an image on the retina 41 through the focus adjustment optical system 39, the catadioptric optical system 30, the cornea 42, and the crystalline lens 43. Since the pixel PIX and the fundus are conjugated, the infrared light (reflected light Lr) from the fundus forms an image on the pixel through the crystalline lens 43, the cornea 42, the catadioptric optical system 30, and the focus adjustment optical system 39. Consequently, the focus can be adjusted even in the imaging mode. When the operations of the optical unit 85 and the optical unit 86 are interchanged, image capturing for the other of the eyes can be performed.

The infrared light source used for image capturing is included in the pixel PIX in the above structure; however, one embodiment of the present invention is not limited thereto. For example, a light source included in an eye tracking camera system can also be used as the infrared light source while the pixel PIX that does not include the subpixel 23 exhibiting infrared light is used in the display panel 20. The operation in which one of the optical units 85 and 86 is in the display mode and the other is in the imaging mode are common to this example and the above structure.

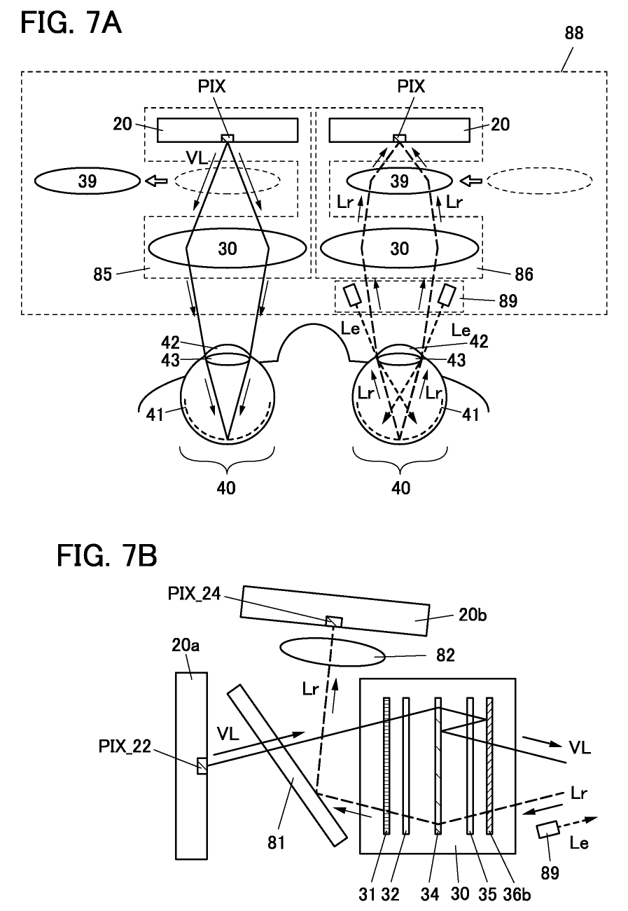

As described above, the pixel density of the display panel used for VR applications is thousands of ppi, and thus the sufficient area for subpixels for lighting cannot be ensured in some cases. In that case, for example, an infrared light source of an eye tracking camera system 89 provided as an independent unit in the electronic device 88 can be used as a light source for fundus image capturing, as illustrated in FIG. 7A.

Light emitted from the infrared light source included in the eye tracking camera system 89 does not necessarily form an image on the fundus, and fundus image capturing can be performed when the reflected light forms an image on the pixel PIX. Thus, the infrared light source can be provided outside the display panel and the optical system. Note that the subpixel 23 and the infrared light source included in the eye tracking camera system 89 can be used together as infrared light sources with the use of the display panel 20 including the subpixel 23.

In the case where the infrared light source included in the eye tracking camera system 89 is used, it is possible to employ a structure where a display panel 20a, a dichroic mirror 81, and an imaging device 20b are used as illustrated in FIG. 7B. The display panel 20a replaces the display panel 20 and includes a pixel PIX_22 including a light-emitting element for display. The dichroic mirror 81 is provided between the display panel 20a and the catadioptric optical system 30. The imaging device 20b includes a pixel PIX_24 including a light-receiving element and is provided in the direction in which the reflected light Lr is reflected. A focus adjustment optical system 82 is provided on an optical path from the dichroic mirror 81 to the imaging device 20b.

As the dichroic mirror 81, a component that transmits the visible light VL and can reflect infrared light is used. Accordingly, the visible light VL (to form an image) emitted from the pixel PIX_22 of the display panel 20a can form an image on the user's eye through the dichroic mirror 81 and the catadioptric optical system 30. That is, the user can view the image on the display panel 20a.

The reflected light Lr of the infrared light (emission light Le) emitted from the eye tracking camera system 89 which is reflected from the fundus passes through the catadioptric optical system 30 and is reflected by the dichroic mirror 81. Then, the reflected light Lr passes through the focus adjustment optical system 82 and forms an image on the pixel PIX_24 included in the imaging device 20b. That is, a fundus image of the user can be captured by the imaging device 20b. With such a structure, the emission efficiency of the display panel 20a and the light-receiving efficiency of the imaging device 20b can be improved, so that image capturing with a high S/N ratio can be performed.

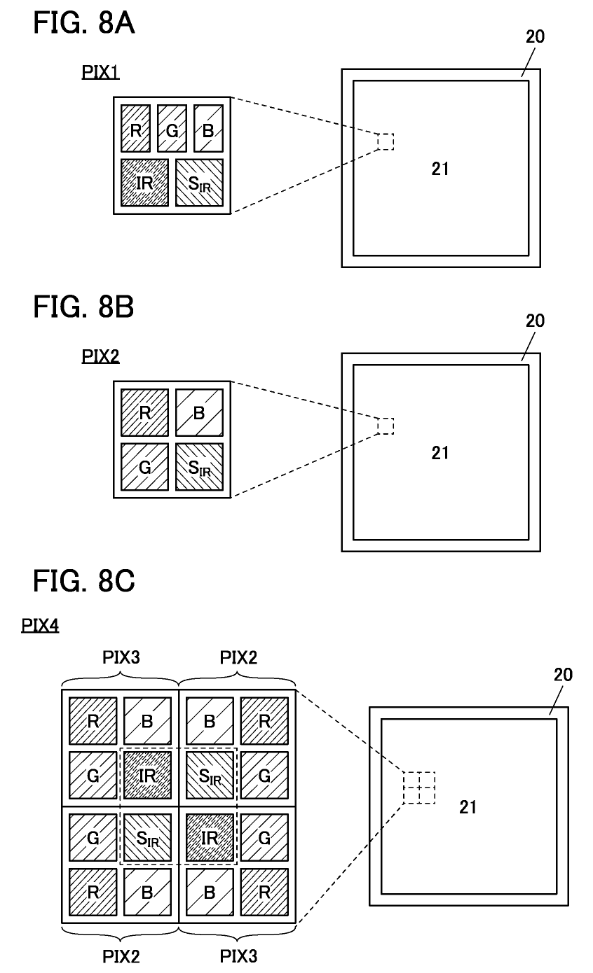

Next, structure examples of pixels that can be used for the display panel of one embodiment of the present invention are described. FIG. 8A illustrates a pixel PIX1 that can be used as the pixel PIX included in the display portion 21 of the display panel 20. The pixel PIX1 can be used as the pixel PIX illustrated in FIG. 1, FIG. 6, and the like.

The pixel PIX1 includes a subpixel R including a light-emitting element emitting red light, a subpixel G including a light-emitting element emitting green light, and a subpixel B including a light-emitting element emitting blue light. In the pixel PIX1, the subpixels R, G, and B can each emit the light (visible light VL) performing color display.

In addition, the pixel PIX1 includes a subpixel IR including a light-emitting element that emits the illumination light (emission light Le) for image capturing, and a subpixel S_{IR} including a light-receiving element that performs photoelectric conversion of the infrared light (reflected light Lr) reflected by an object. The subpixels R, G, and B each correspond to the subpixel 22 illustrated in FIG. 1, the subpixel IR corresponds to the subpixel 23 illustrated in FIG. 1, and the subpixel S_{IR} corresponds to the subpixel 24 illustrated in FIG. 1. Note that the shapes and sizes of the subpixels R, G, B, IR, and S_{IR} in the top view can be adjusted as appropriate in accordance with the emission efficiency of each light-emitting element, the light-receiving sensitivity of the light-receiving element, and the like.

The pixel PIX1 includes a large number of subpixels, which leads to the difficulty in increasing the sensitivity of sensing. Meanwhile, each pixel includes the subpixel including the light-receiving element and therefore achieves image capturing without reducing definition.

FIG. 8B illustrates a pixel PIX2, which has a structure different from that in FIG. 8A and can be used as the pixel PIX illustrated in FIG. 7A and the like. The pixel PIX2 includes the subpixels R, G, B and S_{IR}. The pixel PIX2 is different from the pixel PIX1 in not including the subpixel IR.

The pixel PIX2 needs an infrared light source for lighting provided outside the display panel 20, which might increase the number of components of the electronic device. Meanwhile, the area required for the subpixel IR can be assigned to another subpixel, and accordingly the display quality and the sensitivity of sensing can be improved.

FIG. 8C illustrates an example in which a pixel unit PIX4 having a structure different from those in FIGS. 8A and 8B is provided in the display portion 21. The pixel unit PIX4 includes the pixel PIX2 and a pixel PIX3. The pixel PIX3 includes the subpixels R, G, B and IR. The pixel PIX3 is different from the pixel PIX1 in not including the subpixel S_{IR}.

FIG. 8C illustrates a structure example in which the pixel PIX2 and the pixel PIX3 are alternately arranged and the subpixel arrangements are varied as appropriate. In such an arrangement example, the subpixel S_{IR} included in the pixel PIX2 and the subpixel IR included in the pixel PIX3 can be adjacent to each other. Thus, although the subpixel IR and the subpixel S_{IR} are not present in the same pixel, the subpixel S_{IR} is easily within the spot diameter of the reflected light Lr (see FIG. 5A).

In the pixel unit PIX4, the subpixel IR and the subpixel S_{IR} are placed in two different pixels, and accordingly the imaging definition decreases more than in the structure using the pixel PIX1. Meanwhile, the areas of the subpixels R, G, and B can be increased, leading to higher display quality.

Next, a specific structure of the catadioptric optical system 30 and details of the polarization state are described.

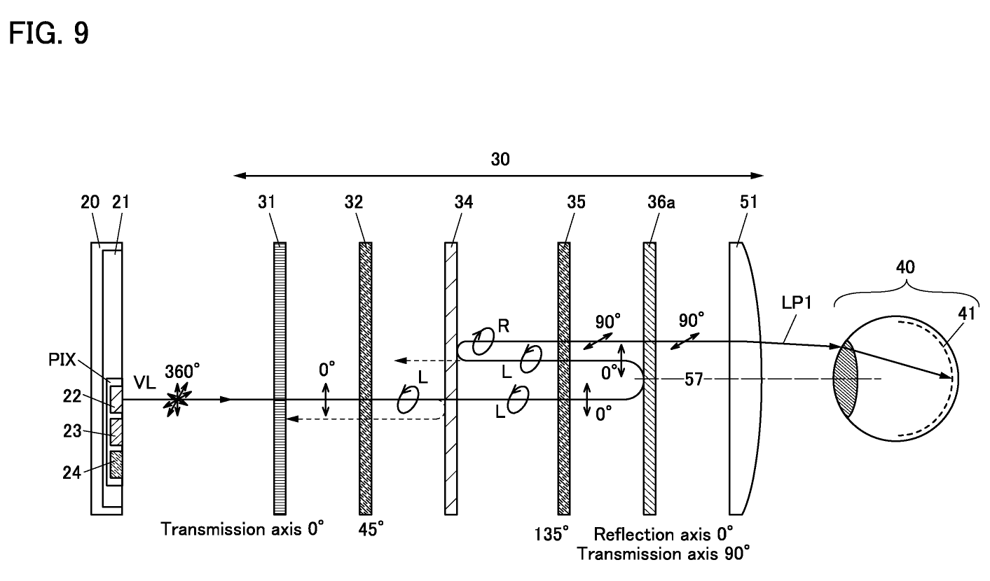

FIG. 9 illustrates the display panel 20 and the catadioptric optical system 30 included in the electronic device. FIG. 9 also illustrates an optical path LP1 of light that is emitted from the subpixel 22 of the pixel PIX and reaches the fundus including the retina 41 of the eye 40. The optical path LP1 is the normal optical path of the catadioptric optical system 30. Note that the shapes and positions of the components illustrated in FIG. 9 are examples.

In the catadioptric optical system 30, the linear polarizing plate 31, the retardation plate 32, the half mirror 34, the retardation plate 35, the reflective polarizing plate 36, and a lens 51 are arranged in one direction in this order from the display panel 20 side, and the optical axis 57 passes through their centers. Note that each combination of the polarizing plate and the retardation plate (the combination of the linear polarizing plate 31 and the retardation plate 32 and that of the retardation plate 35 and the reflective polarizing plate 36) is also referred to as a circularly polarizing plate, which converts non-polarized light into circularly polarized light.

Although FIG. 9 illustrates an example where the lens 51 is positioned between the reflective polarizing plate 36 and the eye 40, one embodiment of the present invention is not limited thereto. The lens 51 can be provided at another position, or a plurality of lenses including the lens 51 can be provided. The lens 51 or the like can be used also as a support of another component of the catadioptric optical system 30. When the lens 51 is used as a support, components of the optical system can be reduced.

Although the components of the catadioptric optical system 30 are illustrated in FIG. 9 to be apart from each other for clear description of the optical path and the polarization state, one embodiment of the present invention is not limited thereto. Some adjacent components can be placed proximate to each other. Proximately placing adjacent components can reduce the interface between the air and the components, which may prevent unnecessary reflection.

To be proximately placed, for example, adjacent components can be in contact with each other by optical contact without any adhesive or the like therebetween. This makes an adhesive unnecessary, leading to higher heat resistance and higher chemical resistance. Furthermore, the elimination of the component with a different refractive index leads to prevention of unnecessary reflection.

Alternatively, the components are preferably bonded to each other with an optical adhesive that has high transmittance with respect to the wavelength of light to be used (the wavelength ranges of visible light and infrared light described above in one embodiment of the present invention), no absorption of specific polarized light, and no birefringence. With such a structure, characteristics equivalent to those of the optical contact can be obtained.

Alternatively, one component can be formed on and in contact with the other component by a method such as coating, not by bonding. Alternatively, a void can be provided between the components. In the structure, the position of the components can be changed on the optical axis, which increases the degree of freedom in design.

An anti-reflection layer can be provided on the surface of a component that transmits light and has an interface with the air. Since unnecessary reflection on a surface of the component (an interface between the air and the component) is prevented, light utilization efficiency can be improved and generation of stray light can be inhibited. Note that an anti-reflection layer is not needed for the half mirror, which also has a reflecting action, and the reflective polarizing plate.

As the anti-reflection layer, a film-shaped anti-reflection film or a dielectric multilayer film can be used. For example, a dielectric multilayer film is preferably provided on a curved surface like a lens surface, to which a film is not easily attached. In the case of a component having a flat surface, either a film-shaped anti-reflection film or a dielectric multilayer film can be provided. Note that in the case where the component in which the anti-reflection layer is formed has a resin film shape or the like, the component is thermally damaged in the formation process of the dielectric multilayer film in some cases. In such a case, a film-shaped anti-reflection film is preferably provided over the component with an adhesive therebetween.

There are a film-shaped anti-reflection film of a type that cancels light by interference of reflected light and a film-shaped anti-reflection film of a moth-eye type in which minute projections formed on a surface cause a continuous change in the refractive index. Whichever type is employed, a film that is not formed by a stretching process is preferably used as a base film. A film formed by a stretching process has optical anisotropy in some cases and thus may change the polarization state in some cases. It can be said that a moth-eye type is preferably used because of its small angular dependence and small wavelength dependence.

With use of the catadioptric optical system 30 having such a structure, light emitted from the display panel 20 or light reflected by the fundus is converted into linearly polarized light or circularly polarized light to be utilized, whereby reflection and transmission can be selectively performed with a component placed on the optical path. Thus, the optical path length can be ensured in a limited space, and the catadioptric optical system 30 can be downsized.

Next, the components of the display panel 20 and the catadioptric optical system 30 are described in detail.

As the display panel 20, a liquid crystal panel including a liquid crystal element, an organic EL panel including an organic EL element, an LED panel including a micro LED, or the like can be used. In particular, an organic EL panel is preferably used because a self-luminous and high-resolution display portion is easily formed. In this specification and the like, a light-emitting diode whose chip area is less than or equal to 10000 μm² is referred to as a micro LED. Note that the LED panel is not limited to the micro LED; a light-emitting diode whose chip area is greater than 10000 μm² and less than or equal to 1 mm² (also referred to as a mini LED) can be used, for example. Increasing pixel size makes the manufacturing process easier when image quality is acceptable. In this embodiment, an example where an organic EL panel including the light-receiving element is used is described. Note that the display panel used in the goggles-type device is small and has high resolution, and for example, a panel with a diagonal size of 0.5 inches to 1.5 inches is preferably used.

The linear polarizing plate 31 can transmit one linearly polarized light from light oscillating in 360° all directions (non-polarized light). As the linear polarizing plate 31, a thin film with uniaxial alignment of iodine or dye, a wire grid polarizing plate, or a dielectric multilayer film can be used, for example.

Although description is given here on the assumption that the transmission axis of the linear polarizing plate 31 is 0°, 0° is not an absolute value, but a reference value. That is, the polarization plane of the linearly polarized light passing through the linear polarizing plate 31 is regarded as 0° Accordingly, for example, 90° linearly polarized light in this embodiment refers to linearly polarized light obtained by rotating the polarization plane of the linearly polarized light passing through the linear polarizing plate 31 by 90°.

The retardation plate 32 has a function of converting linearly polarized light into circularly polarized light. Here, a λ/4 plate (a quarter-wave plate) is used as the retardation plate 32. The λ/4 plate is overlaid with the linear polarizing plate 31 such that the angle of the slow axis of the λ/4 plate with respect to the axis of the linearly polarized light extracted by the linear polarizing plate 31 becomes 45°, whereby a right-handed circularly polarized light (right circularly polarized light) is obtained. The λ/4 plate is overlaid with the linear polarizing plate 31 such that the angle of the slow axis of the λ/4 plate with respect to the axis of the linearly polarized light extracted by the linear polarizing plate 31 becomes -45° (135°), whereby a left-handed circularly polarized light (left circularly polarized light) is obtained. In one embodiment of the present invention, either right circularly polarized light or left circularly polarized light can be used as long as combination with the characteristics of the reflective polarizing plate 36 described later is appropriate.

As the half mirror 34, for example, a dielectric multilayer film in which a film with a relatively high refractive index and a film with a relatively low refractive index are alternately stacked can be used. Note that the half mirror refers to a mirror whose reflectance and transmittance are both 50 % in a narrow sense; however, the half mirror in this specification is not limited thereto. The normal optical path of the catadioptric system includes an optical path where light passes through the half mirror and then is reflected.

Thus, the light utilization efficiency is the product of the reflectance and the transmittance; however, since the reflectance + the transmittance equals approximately 1, when one of the reflectance and the transmittance becomes smaller, the other becomes larger. That is, in a normal optical path of a catadioptric optical system where a half mirror is used twice for reflection and transmission, the light utilization efficiency does not change largely even when the reflectance and the transmittance are each deviated from 50 %. Accordingly, the reflectance and transmittance of the half mirror used in the catadioptric optical system are each not limited to 50 % and can be, for example, 40 % to 60 % in the wavelength range of visible light. Note that in one embodiment of the present invention, the optical path that includes the transmission path as a main part and from which the reflection path is omitted is also used; therefore, the transmittance of the half mirror 34 is preferably higher than or equal to 50 %.

Examples of a material with a low refractive index that can be used for the dielectric multilayer film include silicon oxide, silicon oxynitride, magnesium fluoride, lithium fluoride, and sodium fluoride. Examples of a material with a high refractive index that can be used for the dielectric multilayer film include titanium oxide, niobium oxide, silicon nitride, aluminum oxide, zirconium oxide, and hafnium oxide. As the material that can be used for the dielectric multilayer film, a material with a low extinction coefficient is preferably selected to minimize optical loss.

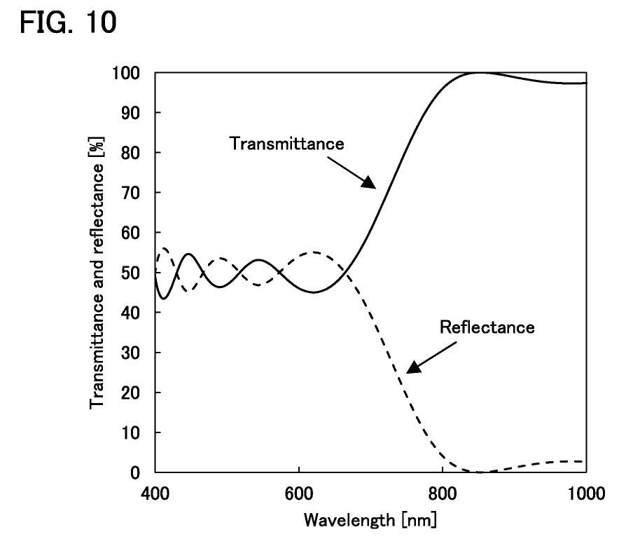

In order to increase the efficiency of utilizing infrared light (the emission light Le and the reflected light Lr) emitted from the subpixel 23, it is preferable that the half mirror 34 partially transmit and partially reflect visible light visible light and transmit infrared light, as described above. With the structure where the material with a low refractive index and the material with a high refractive index described above are stacked appropriately, it is possible to form the half mirror having the characteristics shown in FIG. 10 that partially transmits and partially reflects visible light and transmits infrared light, for example.

Note that in this specification and the like, the term "transmit" may include some degree of transparency other than full transparency. For example, FIG. 10 shows that the characteristics of the dielectric multilayer film that transmits more than or equal to 95 % of light with a wavelength longer than 800 nm, for example. It can be said that light that has not been transmitted is partly reflected and the rest is absorbed. Thus, although complete transmission is not achieved, having a substantially "transmitting" property can be recognized as long as the transmitting property offers a sufficient effect while being sufficiently greater than the reflective property or the absorbing property.

FIG. 10 shows simulation results of the spectral transmittance and the spectral reflectance of a model formed of glass, the dielectric multilayer film, and the air when light is incident through the air side. As the dielectric multilayer film in the model, 26 stacked films in which SiO₂ and TiO₂ are alternately stacked are used. As simulation software, Essential Macleod (manufactured by Thin Film Center Inc.) is used.

Although not illustrated in FIG. 9, a support is required for the half mirror 34 because the half mirror 34 is an extremely thin dielectric multilayer film. The support is preferably formed using a material having high transmittance of visible light and infrared light, and glass, a resin, or the like can be used.

When the support has a curved surface, the half mirror 34 can have curvature and thus can be used as a concave mirror having positive power. Note that the power refers to the light-gathering power. When the power is positive, the light converges; when the power is negative, the light diverges. The direction and curvature of the concave surface can be set as appropriate in combination with other components included in the catadioptric optical system.

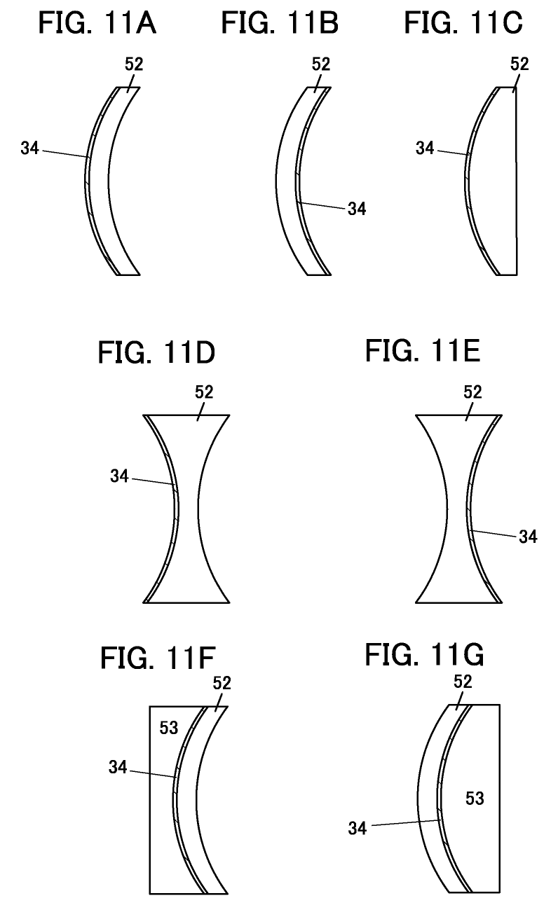

For example, as illustrated in FIGS. 11A and 11B, a support 52 of the half mirror 34 can be formed to have equal curvature on its convex and concave surfaces or have a meniscus lens shape. In this case, the half mirror 34 can function as a concave mirror regardless of whether it is provided on the convex surface or the concave surface.

As illustrated in FIG. 11C, the support 52 can have a convex lens shape. In this case, the half mirror 34 can function as a concave mirror when provided on the convex surface. Although FIG. 11C illustrates the shape of the plano-convex lens as an example, a biconvex lens shape can also be employed.

As illustrated in FIGS. 11D and 11E, the support 52 can have a concave lens shape. Although the shape of the biconcave lens is illustrated as an example in FIGS. 11D and 11E, a plano-concave lens shape can also be employed.

The focal length of the catadioptric optical system can be determined by complex action of the power of the half mirror functioning as a concave mirror and the power of a lens provided in the catadioptric optical system. Thus, as illustrated in FIGS. 11F and 11G, the half mirror 34 can be sandwiched between the support 52 and a support 53 so that a desired focal length can be obtained, for example. When an optically functional layer (an optical film) such as a retardation plate or a polarizing plate is attached to a non-adhesive surface of the support, the number of members can be reduced. Alternatively, the interface between the member and the air can be reduced, so that unnecessary reflection can be prevented.

The supports 52 and 53 can each have a plano-concave shape, a biconcave shape, a plano-convex shape, a biconvex shape, a convex-maniscus shape, or a concave-meniscus shape, and the supports 52 and 53 can have shapes different from each other. The shapes of the supports have features described below, and can be selected as appropriate depending on the purpose.

As an indicator of field curvature, a Petzval sum is given, which is calculated from the refractive indices and focal lengths of the lenses. A Petzval sum of 0 results in a flat image surface, which is a desirable characteristic for a lens system. To achieve this, it is necessary to make either the refractive index or the focal length negative; however, since the refractive index cannot be negative, it is preferable to use a concave lens with a negative focal length. Thus, it can be said that one or more of the supports 52 and 53 preferably have a concave lens shape in order to reduce the field curvature.

Refraction involves chromatic aberration, and combining positive and negative powers is effective for correcting the chromatic aberration. Even if the incident surface is flat, chromatic aberration occurs unless light is perpendicularly incident on the flat surface. Therefore, a combination of surfaces (convex and concave surfaces), the chromatic aberration of one of which is counterbalanced by that of the other, is effective. Thus, it can be said that the supports 52 and 53 are preferably a combination of convex and concave lens shapes to reduce the chromatic aberration.

Simply put, a larger positive power allows for a shorter focal length and a smaller size of the entire optical system. Even in a structure where the half mirror (concave mirror) provides most of the positive power, the focal length can be shortened if there is a convex surface. Thus, it can be said that one or more of the supports 52 and 53 preferably have a convex lens shape to increase the positive power even slightly.

The catadioptric optical system of one embodiment of the present invention needs a polarizing plate and a retardation plate. The polarizing plate and a retardation plate each have a film-like shape in many cases, and their bonding surface is preferably flat in consideration of direct attachment to the support 52 or the support 53. Therefore, in terms of easy manufacturing, it can be said that one or more of the supports 52 and 53 preferably have a flat surface on the outer side.

Although FIGS. 11F and 11G illustrate an example in which the support 52 illustrated in FIGS. 11A and 11B is used, the support 52 illustrated in FIGS. 11C to 11E can also be used.

The retardation plate 35 has a function of reversibly converting linearly polarized light and circularly polarized light. Like the retardation plate 32, the retardation plate 35 can be a λ/4 plate (a quarter-wave plate).

As the reflective polarizing plate 36, it is possible to use a component that transmits infrared light regardless of the polarization state and reflects linearly polarized visible light whose oscillation direction coincides with the reflection axis while transmitting linearly polarized visible light whose oscillation direction is orthogonal to the reflection axis. The reflective polarizing plate having such characteristics can be formed by extending a dielectric multilayer film with different refractive indices, for example.

As the lens 51, a biconvex lens, a plano-convex lens, or a convex meniscus lens can be used, for example. For the lens 51, a structure combining a plurality of lenses selected from a biconvex lens, a plano-convex lens, a convex meniscus lens, a biconcave lens, a plano-concave lens, and a concave meniscus lens can be used depending on the purpose. The lens 51 is not limited to a spherical lens, and may be an aspherical lens. The use of combined lenses or an aspherical lens can reduce a variety of aberrations in the lens. Although not used in the normal optical path, a lens similar to the lens 51 can also be used in the focus adjustment optical system 39.

In the catadioptric optical system 30, a lens made of a resin is desirably used for reduction in weight. On the other hand, a resin has a property of easily having birefringence. A substance with birefringence has varying refractive indices depending on the oscillation direction of polarized light, and thus transmits different polarized components at different speeds. Accordingly, the polarized components after passing through the substance have a phase difference, resulting in change in the polarization state. In the catadioptric optical system, the change in the polarization state causes a light ray deviated from a normal optical path. This light ray enters the eye as stray light and is seen as a double or hazy image.

Although the relationship between the polarization state and the optical path is described later, for the above reasons, a lens on an optical path where polarized light goes back and forth is preferably formed using glass with almost no birefringence. In addition, since a human cannot recognize polarized light, the use of a resin lens with birefringence just before the eye 40 does not adversely affect the visibility of display.

Examples of the resin used for the lens include an acrylic resin, a polycarbonate resin, a polyester resin, and a cycloolefin resin, and these resins can be typically used as a material for the lens. Note that when formed using a material with sufficiently low birefringence, a resin lens can be used on an optical path where polarized light goes back and forth.

Next, the optical path LP1 of the visible light VL emitted from the subpixel 22 illustrated in FIG. 9 is described. The optical path LP1 is the normal optical path of the catadioptric optical system 30 through which an image is formed on the retina 41. The optical path LP1 can be referred to as an optical path in the display mode where an image is viewed.

The linear polarizing plate 31 and the retardation plate 32 transmit part of the light emitted from the subpixel 22 of the pixel PIX, the half mirror 34 partially transmits the light, the retardation plate 35 transmits the light, and the reflective polarizing plate 36 reflects the light. The retardation plate 35 transmits the light reflected by the reflective polarizing plate 36, and the half mirror 34 partially reflects the light. The light reflected by the half mirror 34 passes through the retardation plate 35, the reflective polarizing plate 36, and the lens 51, and is incident on the eye 40 to form an image on the retina 41.

As described above, reflection and transmission can be selectively performed utilizing linearly polarized light, circularly polarized light, the half mirror, and the reflective polarizing plate. Thus, the optical path length can be ensured in a limited space, whereby the focal length of the optical device can be shortened.

Details of the polarization states in the optical paths are described. Light that is emitted from the subpixel 22 and oscillating in all directions (non-polarized light) is incident on the linear polarizing plate 31. Through the linear polarizing plate 31 whose transmission axis is 0°, 0° linearly polarized light passes. Note that in the case where a liquid crystal panel is used as the display panel 20, the linear polarizing plate 31 can be used as one of a pair of polarizing plates included in the liquid crystal panel.

The 0° linearly polarized light passing through the linear polarizing plate 31 is converted into left circularly polarized light (L) by the retardation plate 32 (45°). The half mirror 34 partially transmits the left circularly polarized light (L), and the light is incident on the retardation plate 35 (135°) and converted into 0° linearly polarized light. At this time, the left circularly polarized light (L) (shown by a dashed line) that is transmitted by the half mirror 34 becomes right circularly polarized light (R) with reversed handedness. The right circularly polarized light (R) is incident on the retardation plate 32 and converted into 90° linearly polarized light, and thus is absorbed by the linear polarizing plate 31. Although an example in which the retardation plate 32 is placed at 45° and light passing through the retardation plate 32 is left circularly polarized is described here, the retardation plate 32 can be placed at 135° so that the light is right circularly polarized.

The 0° linearly polarized light emitted through the retardation plate 35 is reflected by the reflective polarizing plate 36 whose reflection axis is 0°, and the light is incident on the retardation plate 35 and converted into left circularly polarized light (L). The half mirror 34 partially reflects the left circularly polarized light (L), and the light becomes right circularly polarized light (R) with reversed handedness. The right circularly polarized light (R) is incident on the retardation plate 35 and converted into 90° linearly polarized light. The 90° linearly polarized light passes through the reflective polarizing plate 36 whose transmission axis is 90° and the lens 51, and is incident on the eye 40 to reach the fundus.

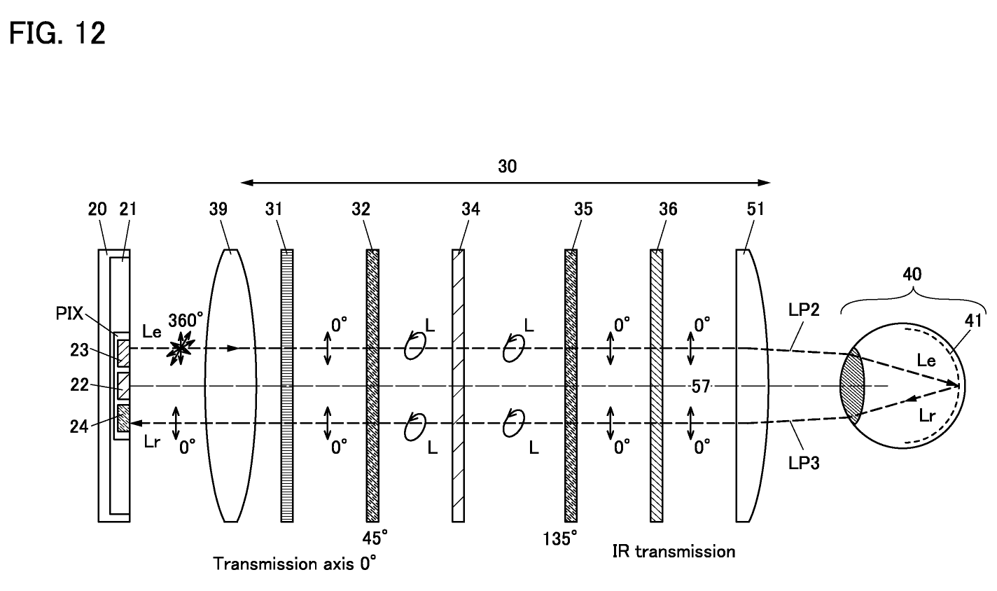

Next, an optical path LP2 of the emission light Le emitted from the subpixel 23 illustrated in FIG. 12 and an optical path LP3 of the reflected light Lr are described. The optical path LP2 is an optical path in which the reflection path of the catadioptric optical system 30 is omitted by the use of infrared light and the focus adjustment optical system 39 is inserted to form an image on the retina 41. The optical path LP3 is an optical path through which light reflected by the retina 41 forms an image on the subpixel 24. The optical paths LP2 and LP3 are substantially equivalent in consideration of the description of FIG. 5A. The optical paths LP2 and LP3 can each be regarded as an optical path in the imaging mode in which an image capturing operation of the fundus is performed.

The route of the optical path LP2 is as follows. Part of infrared light emitted from the subpixel 22 of the pixel PIX passes through the focus adjustment optical system 39, the linear polarizing plate 31, the retardation plate 32, the half mirror 34, the retardation plate 35, the reflective polarizing plate 36, and the lens 51, is incident on the eye 40, and forms an image with on the fundus (retina 41).

Details of the polarization states in the optical path LP2 are described. The emitted light Le that is emitted from the subpixel 22 and oscillating in 360° all directions (non-polarized light) is incident on the linear polarizing plate 31. Through the linear polarizing plate 31 whose transmission axis is 0°, 0° linearly polarized light passes. The 0° linearly polarized light passing through the linear polarizing plate 31 is converted into left circularly polarized light (L) by the retardation plate 32 (45°). The left circularly polarized light (L) passes through the half mirror 34, is incident on the retardation plate 35 (135°), and is converted into 0° linearly polarized light. The 0° linearly polarized light emitted from the retardation plate 35 passes through the reflective polarizing plate 36 and the lens 51, and is incident on the eye 40 to reach the fundus.

The route of the optical path LP3 is as follows. Part of light that has passed through the optical path LP2 and been reflected by the fundus passes through the lens 51, the reflective polarizing plate 36, the retardation plate 35, the half mirror 34, the retardation plate 32, the linear polarizing plate 31, and the focus adjustment optical system 39, and forms an image on the subpixel 23.

Details of the polarization states in the optical path LP3 are described. The 0° linearly polarized component reflected by the fundus passes through the lens 51 and the reflective polarizing plate 36 whose transmissive axis is 0°, is incident on the retardation plate 35, and is converted into left circularly polarized light (L). The left circularly polarized light (L) passes through the half mirror 34, is incident on the retardation plate 32, and is converted into 0° linearly polarized light. The 0° linearly polarized light passes through the linear polarizing plate 31 whose transmissive axis is 0°, and is incident on the subpixel 23.

In this manner, light emitted from the subpixel 23 of the pixel PIX and reflected by the fundus can be incident on the subpixel 24 of the pixel PIX, whereby fundus image capturing can be performed.

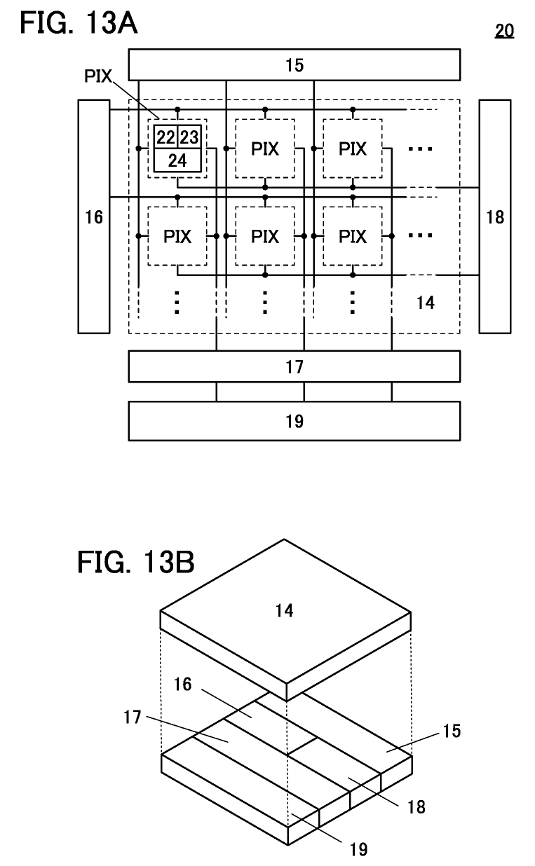

FIG. 13A illustrates the display panel 20 included in the electronic device of one embodiment of the present invention. The display panel 20 includes a pixel array 14, a circuit 15, a circuit 16, a circuit 17, a circuit 18, and a circuit 19. The pixel array 14 includes pixels PIX arranged in a column direction and a row direction.

The pixel PIX can include the subpixels 22, 23, and 24 illustrated in FIG. 1. For example, the subpixel 22 has a function of emitting visible light for display. The subpixel 23 has a function of detecting infrared light for image capturing. The subpixel 24 has a function of detecting light delivered to the display panel 20.

Note that in this specification, although a minimum unit in which independent operation is performed in one "pixel" is defined as a "subpixel" in the description for convenience, a "pixel" may be replaced with a "region" and a "subpixel" may be replaced with a "pixel".

The subpixel 22 includes a light-emitting element that emits visible light. The subpixel 23 includes a light-emitting element that emits infrared light. As these light-emitting elements, EL elements such as organic light-emitting diodes (OLEDs) or quantum-dot light-emitting diodes (QLEDs) are preferably used. Examples of a light-emitting substance contained in the EL element include a substance exhibiting fluorescence (a fluorescent material), a substance exhibiting phosphorescence (a phosphorescent material), a substance exhibiting thermally activated delayed fluorescence (a thermally activated delayed fluorescent (TADF) material), and an inorganic compound (e.g., a quantum dot material). Alternatively, a light-emitting diode (LED) such as a micro-LED can be used as the light-emitting element.

The subpixel 24 includes a light-receiving element that has sensitivity to infrared light. A photoelectric conversion element that detects incident light and generates electric charge can be used as the light-receiving element. The amount of electric charge generated by the light-receiving element depends on the amount of light incident on the light-receiving element. As the light-receiving element, a PN photodiode or a PIN photodiode can be used, for example.

It is preferable to use an organic photodiode containing an organic compound in a photoelectric conversion layer as the light-receiving element. An organic photodiode is easily made thin and lightweight and easily has a large area. In addition, an organic photodiode can be used in a variety of display panels because of its high flexibility in shape and design. Alternatively, a photodiode containing crystalline silicon (single crystal silicon, polycrystalline silicon, microcrystalline silicon, or the like) can be used as the light-receiving element.

In one embodiment of the present invention, an organic EL element is used as the light-emitting element, and an organic photodiode is used as the light-receiving element. The organic photodiode and the organic EL element can share a component. Accordingly, the light-receiving element can be incorporated in the display panel 20 without a significant increase in the number of manufacturing steps. For example, a photoelectric conversion layer of the light-receiving element and a light-emitting layer of the light-emitting element are separately formed, and the other layers can be shared by the light-emitting element and the light-receiving element.

The circuits 15 and 16 are driver circuits for driving the subpixel 22. The circuit 15 can have a function of a source driver and the circuit 16 can have a function of a gate driver. A shift register circuit or the like can be used as each of the circuits 15 and 16, for example. Note that the driver circuits for the subpixels 22 and 23 can be separated.

The circuits 17 and 18 are driver circuits for driving the subpixel 23. The circuit 17 can have a function of a column driver and the circuit 18 can have a function of a row driver. A shift register circuit, a decoder circuit, or the like can be used as each of the circuits 17 and 18, for example.

The circuit 19 is a read circuit for data output from the subpixel 23. The circuit 19 includes, for example, an A/D converter circuit and has a function of converting analog data output from the subpixel 23 into digital data. In addition, the circuit 19 can include a CDS circuit that performs correlated double sampling processing on output data.

As illustrated in FIG. 13B, a structure where the circuits 15 to 19 overlap with the pixel array 14 may be employed. This structure can narrow a bezel of the display panel. The driver circuits are below the pixel array 14, which enables reductions in the wiring lengths, wiring capacitance, and the like. Accordingly, a display panel capable of high-speed operation with low power consumption can be provided. The layouts and areas of the circuits 15 to 19 illustrated in FIG. 13B are examples, and can be changed as appropriate. In addition, part of each of the circuits 15 to 19 can be formed in the same layer as the pixel array 14.

To obtain the above structure, for example, the circuits 15 to 19 can be formed using transistors formed on a single crystal silicon substrate or the like (hereinafter, Si transistors), and a pixel circuit in the pixel array 14 can be formed using transistors each including a metal oxide in a channel formation region (hereinafter, OS transistors). An OS transistor can be formed with a thin film and can be stacked over a Si transistor.

Note that the light-receiving element included in the subpixel 24 can be used not only for fundus image capturing but also for obtaining image data on blinks, eye movement, change in pupil diameter, or the like. By analysis of the image data, gaze detection can be performed. The gaze detection can lead to an input interface function. Alternatively, foveated rendering or the like can also be employed. Furthermore, image capturing data on an iris or the like can be obtained with use of the light-receiving element. That is, a biological authentication function can be added to the display panel.

Next, examples of a pixel circuit of the subpixel including the light-emitting element and a pixel circuit of the subpixel including the light-receiving element are each described.

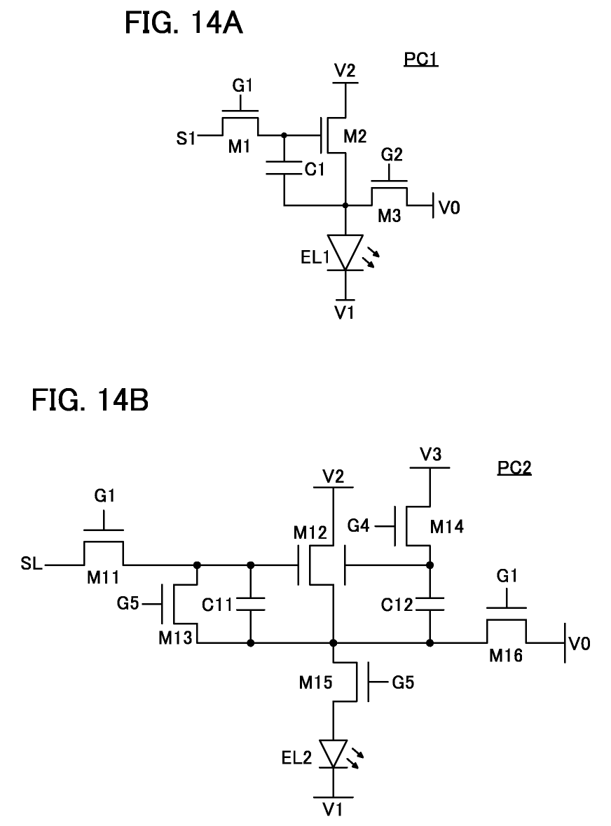

FIG. 14A illustrates an example of a pixel circuit PC1 that can be used for the subpixel 22. The pixel circuit PC1 is a 3Tr1C-type pixel circuit including a light-emitting element EL1, a transistor M1, a transistor M2, a transistor M3, and a capacitor C1. In the example here, a light-emitting diode is used as the light-emitting element EL1. An organic EL element that emits visible light is preferably used as the light-emitting element EL1.

A gate of the transistor M1 is connected to a wiring G1, one of a source and a drain of the transistor M1 is connected to a wiring S1, and the other of the source and the drain of the transistor M1 is connected to one electrode of the capacitor C1 and a gate of the transistor M2. One of a source and a drain of the transistor M2 is connected to a wiring V2, and the other of the source and the drain of the transistor M2 is connected to the other electrode of the capacitor C1, an anode of the light-emitting element EL1, and one of a source and a drain of the transistor M3. A gate of the transistor M3 is connected to a wiring G2, and the other of the source and the drain of the transistor M3 is connected to a wiring V0. A cathode of the light-emitting element EL1 is connected to a wiring V1.

A constant potential is supplied to each of the wirings V1 and V2. Light emission can be performed when the anode side of the light-emitting element EL1 is set to a high potential and the cathode side of the light-emitting element EL1 is set to a low potential. The transistor M1 is controlled by a signal supplied to the wiring G1 and functions as a selection transistor for controlling the selection state of the pixel circuit PC1. The transistor M2 functions as a driving transistor that controls a current flowing through the light-emitting element EL1 in accordance with a potential supplied to the gate of the transistor M2.

When the transistor M1 is on, a potential supplied to the wiring S1 is supplied to the gate of the transistor M2, and the emission luminance of the light-emitting element EL1 can be controlled in accordance with the potential. The transistor M3 is controlled by a signal supplied to the wiring G2. Accordingly, a potential between the transistor M3 and the light-emitting element EL1 can be reset to a constant potential supplied from the wiring V0; thus, a potential can be written to the gate of the transistor M2 in a state where a source potential of the transistor M2 is stabilized.

FIG. 14B illustrates a configuration example different from that of the pixel circuit PC1. Note that some components and wirings connecting them that are common to the pixel circuit PC1 are denoted by the same reference numerals, and the description thereof is omitted. The pixel circuit PC2 includes a transistor M11, a transistor M12, a transistor M13, a transistor M14, a transistor M15, a transistor M16, a capacitor C11, and a capacitor C12. The pixel circuit PC2 is a 6Tr2C-type pixel circuit including six transistors and two capacitors.

The anode of the light-emitting element EL2 is connected to one of a source and a drain of the transistor M15. A cathode of the light-emitting element EL2 is connected to the wiring V1. The other of the source and the drain of the transistor M15 is connected to one of a source and a drain of the transistor M12, one of a source and a drain of the transistor M13, one of a source and a drain of the transistor M16, one electrode of the capacitor C11, and one electrode of the capacitor C12. A gate of the transistor M12 is connected to one of a source and a drain of the transistor M11, the other of the source and the drain of the transistor M13, and the other electrode of the capacitor C11. A back gate of the transistor M12 is connected to one of a source and a drain of the transistor M14 and the other electrode of the capacitor C12.

The other of the source and the drain of the transistor M11 is connected to a wiring SL. The other of the source and the drain of the transistor M12 is connected to a wiring V2. The other of the source and the drain of the transistor M14 is connected to a wiring V3. The other of the source and the drain of the transistor M16 is connected to a wiring V0. The gate of the transistor M11 and the gate of the transistor M16 are connected to the wiring G1. The gate of the transistor M13 and the gate of the transistor M14 are connected to the wiring G4. A gate of the transistor M15 is connected to the wiring G5.

The transistor M11 functions as a selection transistor that controls electrical continuity and discontinuity between the gate of the transistor M12 and the wiring SL. The transistor M12 functions as a driving transistor controlling current flowing through the light-emitting element EL2. The transistor M14 has a function of changing a potential supplied from the wiring V3 to a potential corresponding to the threshold voltage of the transistor M12 and supplying the potential to the back gate of the transistor M12.

When a constant potential is supplied to the back gate of the transistor M12, the threshold voltage can be controlled. The capacitor C11 has a function of retaining a gate potential (a potential difference between the gate and the source) of the transistor M12. The capacitor C12 has a function of retaining a back gate potential (a potential difference between the back gate and the source) of the transistor M12.

The pixel circuit PC2 has a function of correcting the threshold voltage of the transistor M12 with use of the back gate thereof, what is called an internal correcting function of the threshold voltage. Specifically, a back gate potential that makes the threshold voltage of the transistor M12 be 0 V is retained in the capacitor C12. Accordingly, regardless of a variation in the threshold voltage and over-time deterioration of the transistor, the threshold voltage of the transistor M12 can be corrected to be constant at 0 V or the vicinity thereof.

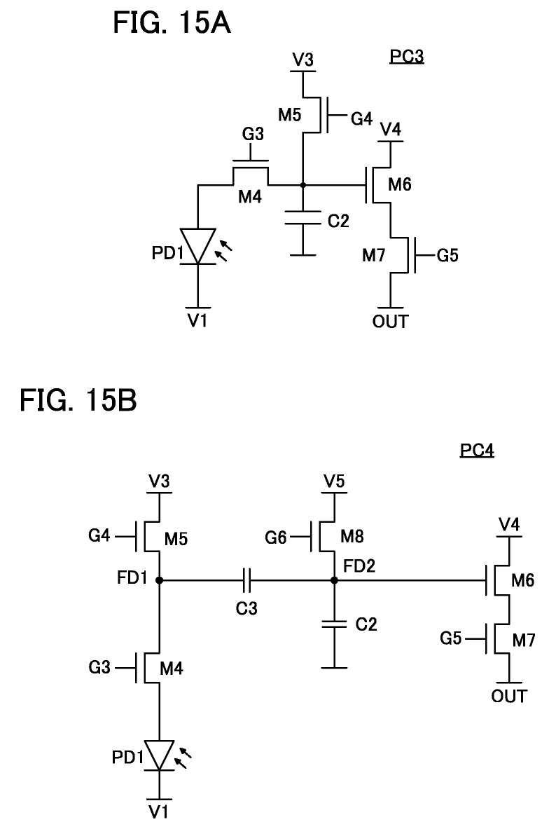

FIG. 15A illustrates an example of a circuit PC3 that can be used for the subpixel 23. The pixel circuit PC3 includes a light-receiving element PD1, a transistor M4, a transistor M5, a transistor M6, a transistor M7, and a capacitor C2. Here, a photodiode is used as an example of the light-receiving element PD1.

A cathode of the light-receiving element PD1 is connected to the wiring V1, and an anode of the light-receiving element PD1 is connected to one of a source and a drain of the transistor M4. A gate of the transistor M4 is connected to a wiring G3, and the other of the source and the drain of the transistor M4 is connected to one electrode of the capacitor C2, one of a source and a drain of the transistor M5, and a gate of the transistor M6. A gate of the transistor M5 is connected to a wiring G4, and the other of the source and the drain of the transistor M5 is connected to a wiring V3. One of a source and a drain of the transistor M6 is connected to a wiring V4, and the other of the source and the drain of the transistor M6 is connected to one of a source and a drain of the transistor M7. A gate of the transistor M7 is connected to a wiring G5, and the other of the source and the drain of the transistor M7 is connected to a wiring OUT.

A constant potential is supplied to each of the wirings V1, V3, and V4. When the light-receiving element PD1 is driven with a reverse bias, the wiring V3 is supplied with a potential lower than the potential of the wiring V1. The transistor M5 is controlled by a signal supplied to the wiring G5 and has a function of resetting the potential of a node connected to the gate of the transistor M6 to a potential supplied to the wiring V3. The transistor M4 is controlled by a signal supplied to the wiring G3 and has a function of controlling the timing at which the potential of the node changes, in accordance with a current flowing through the light-receiving element PD1. The transistor M6 functions as an amplifier transistor outputting a signal corresponding to the potential of the node. The transistor M7 is controlled by a signal supplied to the wiring G6 and functions as a selection transistor for reading the output corresponding to the potential of the node by an external circuit connected to the wiring OUT.

As a modification example of the circuit PC3, a circuit PC4 illustrated in FIG. 15B can also be used. The circuit PC4 is different from the circuit PC3 in including the transistor M13 and a capacitor C3. Note that components common to the circuit PC3 are denoted by the same reference numerals in FIG. 15B.

A cathode of the light-receiving element PD1 is connected to the wiring V1, and an anode of the light-receiving element PD1 is connected to one of a source and a drain of the transistor M4. A gate of the transistor M4 is connected to the wiring G3, and the other of the source and the drain of the transistor M4 is connected to the one electrode of the capacitor C3 and one of the source and the drain of the transistor M5. A gate of the transistor M5 is connected to the wiring G4, and the other of the source and the drain of the transistor M5 is connected to the wiring V3. The other electrode of the capacitor C3 is connected to one of a source and a drain of a transistor M8, the one electrode of the capacitor C2, and the gate of the transistor M6. The one of the source and the drain of the transistor M8 is connected to a wiring V5, and a gate of the transistor M8 is connected to the wiring G6. One of a source and a drain of the transistor M6 is connected to the wiring V4, and the other of the source and the drain of the transistor M6 is connected to one of a source and a drain of the transistor M7. A gate of the transistor M7 is connected to the wiring G5, and the other of the source and the drain of the transistor M7 is connected to the wiring OUT.

Here, a point (e.g., a wiring or an electrode) where the other of the source and the drain of the transistor M4, the one electrode of the capacitor C3, and the one of the source and the drain of the transistor M5 are connected is a node FD1. A point (e.g., a wiring or an electrode) where the other electrode of the capacitor C3, the one of the source and the drain of the transistor M8, the one electrode of the capacitor C2, and the gate of the transistor M6 are connected is referred to as a node FD2.

A constant potential is supplied to each of the wirings V1, V3, V4, and V5. When the light-receiving element PD1 is driven with a reverse bias, the wiring V3 is supplied with a potential lower than the potential of the wiring V1. The transistor M5 is controlled by a signal supplied to the wiring G5 and has a function of resetting the potential of the node FD1 to a potential supplied to the wiring V3. The transistor M8 is controlled by a signal supplied to the wiring G6 and has a function of resetting the node FD2 to a potential supplied to the wiring V5. The transistor M4 is controlled by a signal supplied to the wiring G3 and has a function of controlling the timing at which the potential of the node FD1 changes, in accordance with a current flowing through the light-receiving element PD1. The transistor M6 functions as an amplifier transistor that outputs a signal corresponding to the potential of the node FD2. The transistor M7 is controlled by a signal supplied to the wiring G6 and functions as a selection transistor for reading the output corresponding to the potential of the node by an external circuit connected to the wiring OUT.

In the circuit PC4, the nodes FD1 and FD2 capable of accumulating charge are included, and connected through the capacitor C3. Reset transistors (transistors M4 and M8) that can be reset independently are connected to the nodes FD1 and FD2, respectively. Thus, the initial state can be stored in the node FD2, and a difference between data in the initial state and imaging data stored in the node FD1 can be output.

For example, in the initial state (a first image capturing), an imaging operation is performed in the reset state of the node FD2, and the node FD2 is brought into a floating state, whereby a potential in the initial state can be stored.

Next, only the potential of the node FD1 is reset, and a second image capturing operation is performed. The potential of the node FD2 follows the change in the potential of the node FD1 by capacitive coupling of the capacitor C3. Therefore, when the potential of the node FD1 is the same as that in the initial state in the second and subsequent imaging operations, the potential of the node FD2 does not change; accordingly, a difference of 0 (no change) can be read. When the potential of the node FD1 is different from that in the initial state, the potential of the node FD2 changes by a difference from that in the initial state; accordingly, a change in potential can be read.

That is, when the circuit PC4 is used for the subpixel 23 in one embodiment of the present invention, the presence or absence of change in the eye, the eye's periphery, and the fundus can be read.

A transistor including an oxide semiconductor in a channel formation region (hereinafter, an OS transistor) is preferably used as each of the transistors M1 to M8 and M11 to M16 included in the pixel circuits PC1 to PC4. The OS transistor can have a lower off-state current by using an oxide semiconductor having a wide band gap in a semiconductor layer. The band gap of the oxide semiconductor is preferably greater than or equal to 2 eV, further preferably greater than or equal to 2.5 eV.

There is no particular limitation on the crystallinity of a semiconductor material used in the semiconductor layer of the transistor, and an amorphous semiconductor, a single crystal semiconductor, or a semiconductor having crystallinity other than single crystal (a microcrystalline semiconductor, a polycrystalline semiconductor, or a semiconductor partly including crystal regions) can be used. A single crystal semiconductor or a semiconductor having crystallinity is preferably used, in which case deterioration of the transistor characteristics can be inhibited.

The semiconductor layer provided in the OS transistor preferably contains indium. The semiconductor layer preferably contains indium, M (M is one or more kinds selected from gallium, aluminum, silicon, boron, yttrium, tin, copper, vanadium, beryllium, titanium, iron, nickel, germanium, zirconium, molybdenum, lanthanum, cerium, neodymium, hafnium, tantalum, tungsten, and magnesium), and zinc, for example. Specifically, M is preferably one or more kinds selected from aluminum, gallium, yttrium, and tin.

For example, an oxide containing indium (InOX) is preferably used for the semiconductor layer of the OS transistor. Alternatively, it is preferable to use an oxide containing indium and gallium (also referred to as IGO). Alternatively, it is preferable to use an oxide containing indium and zinc (also referred to as IZO (registered trademark)). Alternatively, it is preferable to use an oxide containing indium, gallium, and zinc (also referred to as IGZO). Alternatively, it is preferable to use an oxide containing indium, tin, and zinc. Alternatively, it is preferable to use an oxide containing indium, gallium, tin, and zinc.

Note that an oxide semiconductor used for the semiconductor layer of the OS transistor is suitably formed by a sputtering method or an ALD method. In the case where the oxide semiconductor is formed by a sputtering method, the productivity and the film density can be increased. In the case where the oxide semiconductor is formed by an ALD method, coverage with a film can be improved.

An OS transistor having a wider band gap and a lower carrier concentration than silicon can achieve an extremely low off-state current. Such a low off-state current enables long-term retention of electric charge accumulated in a capacitor that is connected in series with the transistor.

Thus, it is preferable to use transistors including an oxide semiconductor particularly as the transistors M1, M4, M5, M8, M11, M12, M13, M14, M15 and M16, in each of which one or the other of the source and the drain is connected to the capacitors C1, C2, C3, C11, or C12.

When each of the other transistors also includes an oxide semiconductor, manufacturing cost can be reduced.

Alternatively, transistors using silicon as a semiconductor in which a channel is formed can be used as the transistors M1 to M8 and M11 to M16. In particular, it is preferable to use silicon with high crystallinity such as single crystal silicon or polycrystalline silicon because high field-effect mobility can be achieved and higher-speed operation can be performed.

Alternatively, a transistor using an oxide semiconductor may be used as at least one of the transistors M1 to M8 and M11 to M16, and transistors using silicon may be used as the other transistors.

Note that although FIGS. 14A-14D and 15A-15B each illustrate an example where n-channel transistors are used, p-channel transistors can also be used.

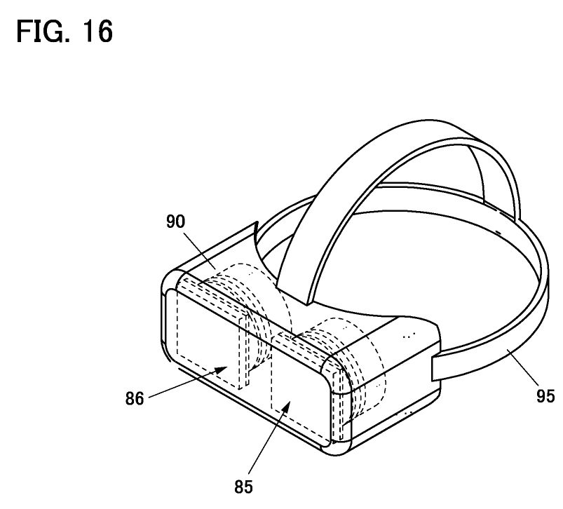

FIG. 16 illustrates an example of the goggles-type device of one embodiment of the present invention. Here, the optical units 85 and 86 illustrated in FIG. 6 are denoted by dashed lines.

A user can see an image displayed on the display panel 20 by bringing their eyes closer to the vicinity of the catadioptric optical system 30 provided on the display surface side of the display panel 20. The user sees the image with a viewing angle widened by the catadioptric optical system 30, and thus can feel a sense of immersion and a realistic sensation.

The optical units 85 and 86 are incorporated in a housing 90. One of the optical units 85 and 86 is for a right eye while the other is for a left eye, and the optical units 85 and 86 each displays an image using parallax, so that the image can be perceived as a three-dimensional image. When one of the optical units 85 and 86 is in the display mode and the other is in the imaging mode as described above, a fundus image can be captured while the eye is focused.

The housing 90 or a support 95 may be provided with an input terminal and an output terminal. To the input terminal, a cable for supplying a video signal from a video output device or the like, power for charging the battery, and the like can be connected. The output terminal can function as, for example, an audio output terminal to which earphones, headphones, or the like can be connected. Note that in the case where audio data can be output by wireless communication or sound is output from an external video output device, the audio output terminal is not necessarily provided.

A wireless communication module, a memory module, and the like may be provided inside the housing 90 or the support 95. A content to be watched can be downloaded via wireless communication using the wireless communication module and stored in the memory module. In this manner, the user can watch downloaded content both offline and online.

The light-emitting element and the light-receiving element included in the display panel 20 can also function as a gaze sensor. In the gaze sensor, light emitted from the light-emitting element is used, and the light-receiving element detects the gaze by reading a change of reflected light due to the movement of the pupil, the iris, and the white of the eye.

For example, a change in reflectance caused by actions of pulling the eyeball over to one of left and right sides or one of the upper and lower sides, and the like is detected, which may be assigned to the operation of the electronic device. For example, operation buttons for power-on, power-off, sleep, volume control, channel change, menu display, selection, decision, and back, and operation buttons for play, stop, pause, fast forward, and rewinding of moving images are displayed and visually recognized, whereby the operations can be performed. The user's fatigue level can be detected from the number of blinks or the like and an alert can be displayed.

The goggles-type device can be used as a health care device for taking fundus photos. A fundus photo can be taken in a mode in which the user operates the goggles-type device as a fundus camera to capture a fundus image. In addition, a fundus photo can also be taken in a mode in which a fundus image is automatically captured when the user is using content on the goggles-type device.

In capturing a fundus photo with the use of the goggles-type device, image data on the fundus can be stored in every use, so that a long-term change in the body can be observed, which can contribute to early detection of diseases and the like. The obtained fundus photo can also be transmitted to a medical institution through a network where the goggles-type device is connected, enabling early stage treatment.

Taking fundus photos leads to the detection of ocular diseases such as glaucoma, macular degeneration, cataracts, fundal hemorrhage, and retinal detachment. The fundus photos can be used for observation of retinal blood vessels, and the conditions of retinal blood vessels enable confirmation of the risk or progression of hypertension, arteriosclerosis, or diabetes. The identification of diseases can effectively use inference by artificial intelligence provided on the goggles-type device or a network connected to the goggles-type device.

At least part of this embodiment can be implemented in combination with any of the other embodiments and an example described in this specification as appropriate.

### Embodiment 2

In this embodiment, structure examples of display devices which can be used for a display panel 20 of one embodiment of the present invention will be described.

The light-emitting element that can be used for the display portion of the display device of one embodiment of the present invention preferably has a metal maskless (MML) structure in which light-emitting layers are separately formed by a lithography process without using a fine metal mask (FMM). A light-emitting element having an MML structure can have a high aperture ratio and emit light with high luminance or low power consumption as compared with a light-emitting element manufactured using an FMM. In addition, a light-emitting element with an MML structure can be combined with a convex lens to further increase light extraction efficiency.

### Display panel 200A

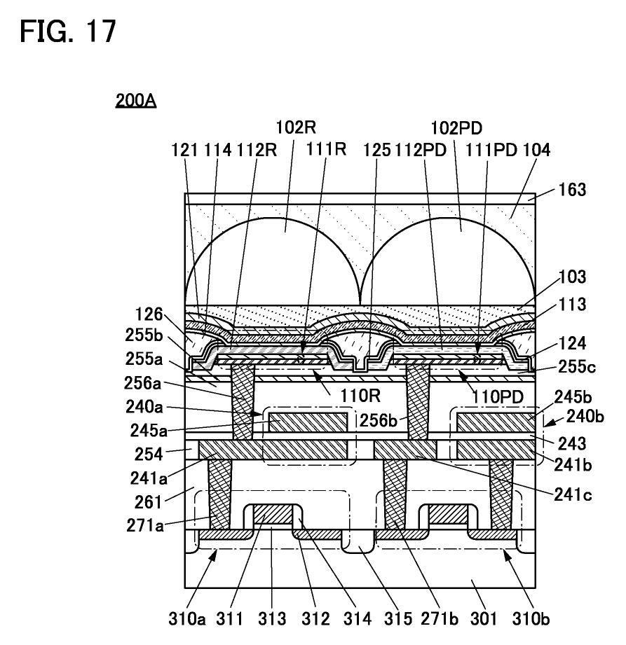

A display panel 200A illustrated in FIG. 17 includes a substrate 301, a light-emitting element 110R, a light-receiving element 110PD, capacitors 240a and 240b, and transistors 310a and 310b. Note that the light-emitting element 110R and the light-receiving element 110PD correspond to, respectively, the light-emitting element included in the subpixel R and the light-receiving element included in the subpixel S illustrated in FIG. 8B, for example. The light-emitting element 110R, the transistor 310a, and the capacitor 240a correspond to, respectively, the light-emitting element EL1, the transistor M1, and the capacitor C1 illustrated in FIG. 14A, for example. The light-receiving element 110PD, the transistor 310b, and the capacitor 240b correspond to, respectively, the light-receiving element PD1, the transistor M4, and the capacitor C2 illustrated in FIG. 15A, for example.

The transistor 310 (transistors 310a and 310b) includes a channel formation region in the substrate 301. As the substrate 301, a semiconductor substrate such as a single crystal silicon substrate can be used, for example. The transistor 310 includes part of the substrate 301, a conductive layer 311, a low-resistance region 312, an insulating layer 313, and an insulating layer 314. The conductive layer 311 functions as a gate electrode. The insulating layer 313 is positioned between the substrate 301 and the conductive layer 311 and functions as a gate insulating layer. The low-resistance region 312 is a region where the substrate 301 is doped with an impurity, and functions as one of a source and a drain. The insulating layer 314 is provided to cover the side surface of the conductive layer 311.

An element isolation layer 315 is provided between two adjacent transistors to be embedded in the substrate 301.

An insulating layer 261 is provided to cover the transistor 310, and the capacitor 240 is provided over the insulating layer 261.

The capacitor 240 (capacitors 240a and 240b) includes a conductive layer 241 (conductive layers 241a and 241b), a conductive layer 245 (conductive layers 245a and 245b), and an insulating layer 243 positioned between the conductive layers 241 and 245. The conductive layer 241 functions as one electrode of the capacitor 240, the conductive layer 245 functions as the other electrode of the capacitor 240, and the insulating layer 243 functions as a dielectric of the capacitor 240.

The conductive layer 241 included in the capacitor 240 is provided over the insulating layer 261 and is embedded in an insulating layer 254. The conductive layer 241 is connected to one of a source and a drain of the transistor 310a or 310b through a plug 271 embedded in the insulating layer 261. The insulating layer 243 is provided to cover the conductive layer 241. The conductive layer 245 is provided in a region overlapping with the conductive layer 241 with the insulating layer 243 therebetween.

An insulating layer 255a is provided to cover the capacitor 240, an insulating layer 255b is provided over the insulating layer 255a, and an insulating layer 255c is provided over the insulating layer 255b.

An inorganic insulating film can be suitably used as each of the insulating layers 255a, 255b, and 255c. For example, it is preferable that a silicon oxide film be used as the insulating layers 255a and 255c and a silicon nitride film be used as the insulating layer 255b. This enables the insulating layer 255b to function as an etching protective film. Although this embodiment describes an example where the insulating layer 255c is partly etched and a depressed portion is formed, the depressed portion is not necessarily provided in the insulating layer 255c.

The light-emitting element 110R and the light-receiving element 110PD are provided over the insulating layer 255c.

As the light-emitting element 110R, an organic light-emitting diode (OLED) or a quantum-dot light-emitting diode (QLED) is preferably used, for example. Examples of the light-emitting substance contained in the EL element include not only organic compounds but also inorganic compounds (e.g., quantum dot materials).

The light-emitting element 110R includes a pixel electrode 111R, an organic layer 112R, a common layer 114, and a common electrode 113.

The pixel electrode111R of the light-emitting element 110R are connected to one of the source and the drain of the transistor 310a through a plug 256a embedded in the insulating layers 255a, 255b, 255c, and 243, the conductive layer 241a embedded in the insulating layer 254, and the plug 271a embedded in the insulating layer 261.

The organic layer 112R included in the light-emitting element 110R contains at least a light-emitting organic compound that emits red light. Each of the organic layer 112R can also be referred to as an EL layer, and includes at least a layer containing a light-emitting substance (a light-emitting layer).

The organic layer 112R and the common layer 114 can independently include one or more of an electron-injection layer, an electron-transport layer, a hole-injection layer, and a hole-transport layer. For example, the organic layer 112R can include a hole-injection layer, a hole-transport layer, a light-emitting layer, and an electron-transport layer that are stacked from the pixel electrode 111R side, and the common layer 114 can include an electron-injection layer.

Each of the common electrode 113 and the common layer 114 is provided as a continuous layer shared by the light-emitting elements. A conductive film having a property of transmitting visible light is used for either the pixel electrodes or the common electrode 113, and a reflective conductive film is used for the other. When the pixel electrodes are light-transmitting electrodes and the common electrode 113 is a reflective electrode, a bottom-emission display device is obtained. Meanwhile, when the pixel electrodes are reflective electrodes and the common electrode 113 is a light-transmitting electrode, a top-emission display device is obtained. Note that when both the pixel electrodes and the common electrode 113 are light-transmitting electrodes, a dual-emission display device is obtained.

The protective layer 121 is provided over the common electrode 113 to cover the light-emitting element 110R. The protective layer 121 has a function of preventing diffusion of impurities such as water into the light-emitting elements from above.

An insulating layer 124, an insulating layer 125, and a resin layer 126 are provided between the light-emitting element and the light-receiving element adjacent to each other or between two light-emitting elements.

The resin layer 126 has a top surface with a smooth convex shape. The top surface of the resin layer 126 is covered with the common layer 114 and the common electrode 113.

The resin layer 126 functions as a planarization film that fills a step between a light-emitting element and a light-receiving element that are adjacent to each other, or between two adjacent light-emitting elements. Providing the resin layer 126 can prevent a phenomenon in which the common electrode 113 is divided by a step at an end portion of the organic layer 112 (also referred to as disconnection) from occurring and the common electrode over the organic layer 112 from being insulated.

The organic layers included in the adjacent light-emitting elements 110 are insulated from each other by the resin layer 126. Accordingly, a leakage current through the organic layers between the adjacent light-emitting elements can be reduced, so that unnecessary light emission due to crosstalk can be inhibited.

An insulating layer containing an organic material can be suitably used as the resin layer 126. Examples of the material used for the resin layer 126 include an acrylic resin, a polyimide resin, an epoxy resin, an imide resin, a polyamide resin, a polyimide-amide resin, a silicone resin, a siloxane resin, a benzocyclobutene-based resin, a phenol resin, and precursors of these resins. The resin layer 126 may be formed using an organic material such as polyvinyl alcohol (PVA), polyvinyl butyral, polyvinylpyrrolidone, polyethylene glycol, polyglycerin, pullulan, water-soluble cellulose, or an alcohol-soluble polyamide resin.

The insulating layer 125 is positioned between the resin layer 126 and the organic layer 112R to function as a protective film for preventing contact between the resin layer 126 and the organic layer 112.

The insulating layer 125 can be an insulating layer containing an inorganic material. As the insulating layer 125, an inorganic insulating film such as an oxide insulating film, a nitride insulating film, an oxynitride insulating film, or a nitride oxide insulating film can be used, for example. The insulating layer 125 may have a single-layer structure or a stacked-layer structure. Examples of the oxide insulating film include a silicon oxide film, an aluminum oxide film, a magnesium oxide film, an indium gallium zinc oxide film, a gallium oxide film, a germanium oxide film, an yttrium oxide film, a zirconium oxide film, a lanthanum oxide film, a neodymium oxide film, a hafnium oxide film, and a tantalum oxide film. Examples of the nitride insulating film include a silicon nitride film and an aluminum nitride film. Examples of the oxynitride insulating film include a silicon oxynitride film and an aluminum oxynitride film. Examples of the nitride oxide insulating film include a silicon nitride oxide film and an aluminum nitride oxide film. In particular, when a metal oxide film such as an aluminum oxide film or a hafnium oxide film or an inorganic insulating film such as a silicon nitride film or a silicon oxide film that is formed by an ALD method is used for the insulating layer 125, the insulating layer 125 has a small number of pin holes and excels in a function of protecting the EL layer.

Note that in this specification and the like, an oxynitride refers to a material in which the oxygen content is higher than the nitrogen content, and a nitride oxide refers to a material in which the nitrogen content is higher than the oxygen content. For example, silicon oxynitride refers to a material that contains more oxygen than nitrogen, and silicon nitride oxide refers to a material that contains more nitrogen than oxygen.

Part of a protective layer (also referred to as a mask layer or a sacrificial layer) for protecting the organic layer 112R during etching of the organic layer 112R survives the etching to become the insulating layer 124. For the insulating layer 124, the material that can be used for the insulating layer 125 can be used. In particular, the insulating layer 124 and the insulating layer 125 are preferably formed using the same material, in which case an apparatus or the like for processing can be used in common.

The protective layer 121 can have, for example, a single-layer structure or a stacked-layer structure at least including an inorganic insulating film. Examples of the inorganic insulating film include oxide films and nitride films such as a silicon oxide film, a silicon oxynitride film, a silicon nitride oxide film, a silicon nitride film, an aluminum oxide film, an aluminum oxynitride film, and a hafnium oxide film. Alternatively, a semiconductor material or a conductive material such as an indium gallium oxide, an indium zinc oxide, an indium tin oxide, or an indium gallium zinc oxide may be used for the protective layer 121.

The insulating layer 103 is provided over the protective layer 121. For the insulating layer 103, an organic material that can be used for the resin layer 126 can be used, for example. The formation of the insulating layer 103 can reduce the influence of an uneven shape due to components below, so that a component such as a lens array can be easily formed.

A plano-convex lens 102R is provided over the insulating layer 103 so as to overlap with the light-emitting element 110R. The insulating layer 104 is provided over the lens 102R.

The lens 102R is provided above the light-emitting element 110R (in the direction in which light is emitted). Since light emitted from the light-emitting element 110R spreads in a certain extent, light not extracted to the outside of the display device is lost. Thus, it is preferable to increase the front luminance of the display device. Since the lens 102R has a convex shape, the lens 102R can lead light to be converged. That is, divergence of the light emitted from the light-emitting element can be inhibited, so that the light extraction efficiency of the display device can be increased.

Using a light-emitting element having higher emission efficiency is also effective for increasing the front luminance of the display panel. In principle, the luminance of a tandem organic EL element increases as the number of stacked units increases at the same current density; the luminance of a two-unit tandem organic EL element can be twice that of a single light-emitting element.

The lifetime of an organic EL element depends on the current density; thus, at the same current density, a tandem organic EL element has an equivalent lifetime to a single organic EL element even when having a doubled luminance. That is, a tandem organic EL element can be regarded as being effective in increasing the luminance and the reliability of an organic EL element.

As the light-receiving element 110PD, a PN photodiode or a PIN photodiode can be used, for example. The amount of electric charge generated from the light-receiving elements 110PD depends on the amount of light entering the light-receiving elements 110PD.

The light-receiving element 110PD can detect visible light. It is particularly preferable to use an organic photodiode including a layer containing an organic compound as the light-receiving element 110PD. An organic photodiode, which is easily made thin, lightweight, and large in area and has a high degree of freedom for shape and design, can be used in a variety of display devices.

In one embodiment of the present invention, organic EL elements are used as the light-emitting elements 110, and organic photodiodes are used as the light-receiving elements 110PD. The organic EL elements and the organic photodiodes can be formed over one substrate. Thus, the organic photodiode can be incorporated in a display device including the organic EL elements.

A manufacturing method similar to that for the light-emitting element 110R can be employed for the light-receiving element 110PD. An island-shaped active layer (also referred to as a photoelectric conversion layer) included in the light-receiving element 110PD is formed by processing a film to be the active layer and formed on the entire surface, not by using a fine metal mask; thus, the island-shaped active layer can have a uniform thickness. Moreover, providing the mask layer over the active layer can reduce damage to the active layer in the manufacturing process of the display device, resulting in an improvement in reliability of the light-receiving element 110PD.

The light-emitting-receiving 111PD includes a pixel electrode 111PD, an organic layer 112PD, the common layer 114, and the common electrode 113.

The pixel electrode 111PD of the light-emitting-receiving 111PD is connected to one of the source and the drain of the transistor 310b through a plug 256b embedded in the insulating layers 255a, 255b, 255c, and 243, the conductive layer 241c embedded in the insulating layer 254, and the plug 271b embedded in the insulating layer 261.

Here, the organic layer 112PD includes at least an active layer, preferably includes a plurality of functional layers. Examples of the functional layer include carrier-transport layers (a hole-transport layer and an electron-transport layer) and carrier-blocking layers (a hole-blocking layer and an electron-blocking layer). In addition, one or more layers are preferably formed over the active layer. A layer between the active layer and the mask layer can inhibit the active layer from being exposed on the outermost surface during the manufacturing process of the display device and can reduce damage to the active layer. Consequently, the reliability of the light-receiving element 110PD can be increased. Thus, the organic layer 112PD preferably includes an active layer and a carrier-blocking layer (a hole-blocking layer or an electron-blocking layer) or a carrier-transport layer (an electron-transport layer or a hole-transport layer) over the active layer.

The organic layer 112PD is provided in the light-receiving element 110PD and is not provided in the light-emitting element 110R. Note that the functional layer other than the active layer in the organic layer 112PD may include the same material as the functional layer other than the light-emitting layer in the light-emitting element 110R. The common layer 114 and the common electrode 113 are each a continuous layer shared by the organic layer 112PD and the light-emitting element 110R.

The function of a layer shared by the light-receiving element 110PD is different from its function in the light-receiving element 110R in some cases. In this specification, the name of a component is based on its function in the light-emitting element 110R. For example, a hole-injection layer functions as a hole-injection layer in the light-emitting element 110R and functions as a hole-transport layer in the light-receiving element 110PD. Similarly, an electron-injection layer functions as an electron-injection layer in the light-emitting element 110R and functions as an electron-transport layer in the light-receiving element 110PD. A layer shared by the light-receiving element 110PD and the light-emitting element 110R may have the same functions in both elements. For example, a hole-transport layer functions as a hole-transport layer in both of the light-emitting element 110R and the light-receiving element 110PD, and an electron-transport layer functions as an electron-transport layer in both of the light-emitting element 110R and the light-receiving element 110PD.

The insulating layer 103 is provided over the common electrode 113. A plano-convex lens 102PD is provided over the insulating layer 103 so as to overlap with the light-receiving element 110PD. A substrate 163 is provided over the lens 102PD with the insulating layer 104 therebetween.

The lens 102PD can lead light to be converged because it has a convex shape. Accordingly, a larger amount of light can be incident on the light-receiving element 110PD, so that the detection sensitivity of the light-receiving element 110PD can be increased.

### Display panel 200B

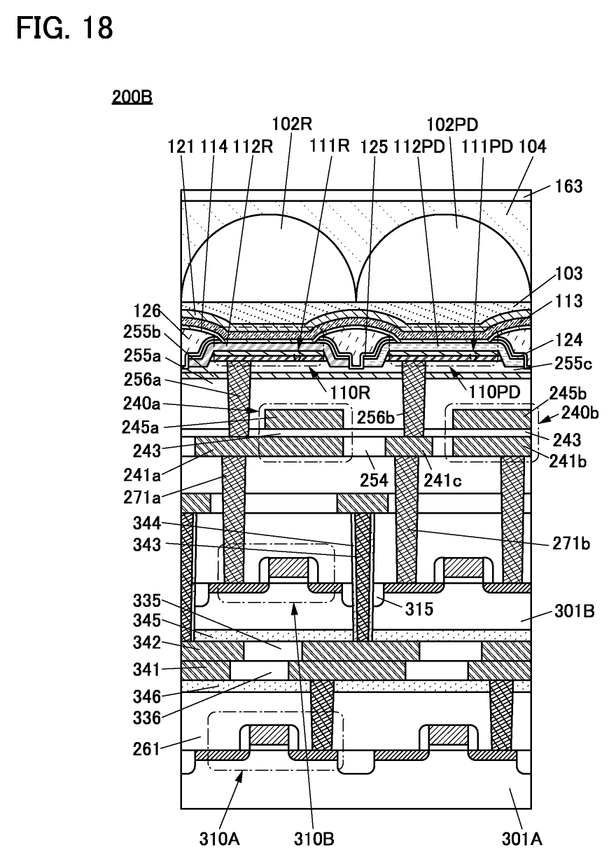

A display panel 200B illustrated in FIG. 18 has a structure in which a transistor 310A and a transistor 310B each having a channel formed in a semiconductor substrate are stacked. Note that in the following description of display panels, the description of portions similar to those of the above-described display panel may be omitted.

In the display panel 200B, a substrate 301B provided with the transistor 310B, the capacitor 240, and the light-emitting devices is attached to a substrate 301A provided with the transistor 310A.

Here, an insulating layer 345 is provided on the bottom surface of the substrate 301B. An insulating layer 346 is provided over the insulating layer 261 over the substrate 301A. The insulating layers 345 and 346 function as protective layers and can inhibit diffusion of impurities into the substrate 301B and the substrate 301A. As the insulating layers 345 and 346, an inorganic insulating film that can be used as the protective layer 121 can be used.

The substrate 301B is provided with a plug 343 penetrating the substrate 301B and the insulating layer 345. An insulating layer 344 functioning as a protective layer is preferably provided to cover the side surface of the plug 343.

A conductive layer 342 is provided under the insulating layer 345 on the rear surface of the substrate 301B. The conductive layer 342 is embedded in an insulating layer 335. The bottom surfaces of the conductive layer 342 and the insulating layer 335 are planarized. The conductive layer 342 is connected to the plug 343.

A conductive layer 341 is provided over the insulating layer 346 over the substrate 301A. The conductive layer 341 is embedded in an insulating layer 336. The top surfaces of the conductive layer 341 and the insulating layer 336 are planarized.

The conductive layers 341 and 342 are preferably formed using the same conductive material. For example, it is possible to use a metal film containing an element selected from Al, Cr, Cu, Ta, Ti, Mo, and W, or a metal nitride film containing any of the above elements as a component (a titanium nitride film, a molybdenum nitride film, or a tungsten nitride film). Copper is particularly preferably used for the conductive layers 341 and 342. In that case, it is possible to employ copper-to-copper (Cu-to-Cu) direct bonding (a technique for achieving electrical continuity by connecting copper (Cu) pads).

### Display panel 200C

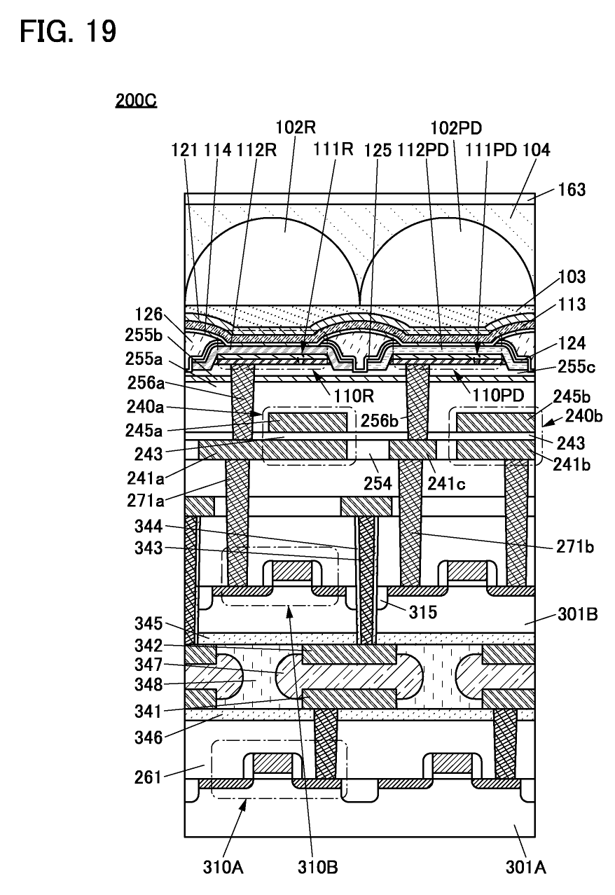

A display panel 200C illustrated in FIG. 19 has a structure in which the conductive layer 341 and the conductive layer 342 are bonded to each other with a bump 347.

As illustrated in FIG. 19, providing the bump 347 between the conductive layers 341 and 342 enables the conductive layers 341 and 342 to be electrically connected to each other. The bump 347 can be formed using a conductive material containing gold (Au), nickel (Ni), indium (In), tin (Sn), or the like, for example. As another example, solder may be used for the bump 347. An adhesive layer 348 may be provided between the insulating layers 345 and 346. In the case where the bump 347 is provided, the insulating layers 335 and 336 may be omitted.

### Display panel 200D

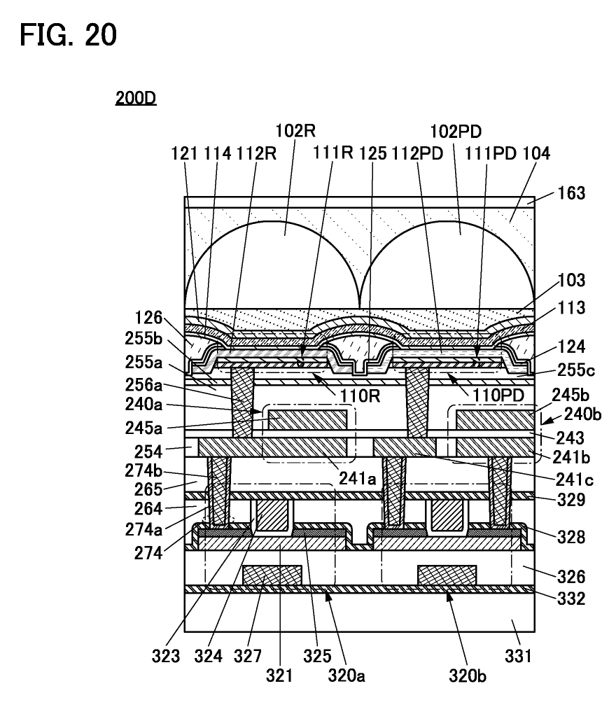

A display panel 200D illustrated in FIG. 20 differs from the display panel 200A mainly in a structure of a transistor.

A transistor 320 (a transistor 320a and a transistor 320b) is a transistor containing a metal oxide (also referred to as an oxide semiconductor) in a semiconductor layer where a channel is formed (i.e., an OS transistor).

The transistor 320 includes a semiconductor layer 321, an insulating layer 323, a conductive layer 324, a pair of conductive layers 325, an insulating layer 326, and a conductive layer 327.

An insulating layer 332 is provided over the substrate 331. The insulating layer 332 functions as a barrier layer that prevents diffusion of impurities such as water or hydrogen from the substrate 331 into the transistor 320 and release of oxygen from the semiconductor layer 321 to the insulating layer 332 side. As the insulating layer 332, for example, a film in which hydrogen or oxygen is less likely to diffuse than in a silicon oxide film, such as an aluminum oxide film, a hafnium oxide film, or a silicon nitride film, can be used.

The conductive layer 327 is provided over the insulating layer 332, and the insulating layer 326 is provided to cover the conductive layer 327. The conductive layer 327 functions as a first gate electrode of the transistor 320, and part of the insulating layer 326 functions as a first gate insulating layer. An oxide insulating film such as a silicon oxide film is preferably used as at least part of the insulating layer 326 which is in contact with the semiconductor layer 321. The top surface of the insulating layer 326 is preferably planarized.

The semiconductor layer 321 is provided over the insulating layer 326. A metal oxide film having semiconductor characteristics (also referred to as an oxide semiconductor film) is preferably used as the semiconductor layer 321. The pair of conductive layers 325 is provided over and in contact with the semiconductor layer 321, and functions as a source electrode and a drain electrode.

An insulating layer 328 is provided to cover the top and side surfaces of the pair of conductive layers 325, the side surface of the semiconductor layer 321, and the like, and an insulating layer 264 is provided over the insulating layer 328. The insulating layer 328 functions as a barrier layer that prevents diffusion of impurities such as water or hydrogen from the insulating layer 264 and the like into the semiconductor layer 321 and release of oxygen from the semiconductor layer 321. As the insulating layer 328, an insulating film similar to the insulating layer 332 can be used.

An opening reaching the semiconductor layer 321 is provided in the insulating layers 328 and 264. The insulating layer 323 that is in contact with the top surface of the semiconductor layer 321 and the conductive layer 324 are embedded in the opening. The conductive layer 324 functions as a second gate electrode, and the insulating layer 323 functions as a second gate insulating layer.

The top surfaces of the conductive layer 324, the insulating layer 323, and the insulating layer 264 are planarized to be level with or substantially level with each other, and insulating layers 329 and 265 are provided to cover these layers.

The insulating layers 264 and 265 function as interlayer insulating layers. The insulating layer 329 functions as a barrier layer that prevents diffusion of impurities such as water or hydrogen from the insulating layer 265 or the like into the transistor 320. As the insulating layer 329, an insulating film similar to the insulating layers 328 and 332 can be used.

A plug 274 connected to one of the pair of conductive layers 325 is provided to be embedded in the insulating layers 265, 329, and 264. Here, the plug 274 preferably includes a conductive layer 274a covering the side surface of an opening formed in the insulating layers 265, 329, 264, and 328 and part of the top surface of the conductive layer 325, and a conductive layer 274b in contact with the top surface of the conductive layer 274a. For the conductive layer 274a, a conductive material in which hydrogen and oxygen are less likely to diffuse is preferably used.

There is no particular limitation on the structure of the transistors included in the display panel of this embodiment. For example, a planar transistor, a staggered transistor, or an inverted staggered transistor can be used. A top-gate transistor or a bottom-gate transistor can be used. Alternatively, gates may be provided above and below a semiconductor layer where a channel is formed.

A structure in which the semiconductor layer where a channel is formed is sandwiched between two gates is employed for the transistor 320. The two gates may be connected to each other and supplied with the same signal to drive the transistor. Alternatively, the threshold voltage of the transistor may be controlled by applying a potential for controlling the threshold voltage to one of the two gates and a potential for driving to the other gate.

An OS transistor has much higher field-effect mobility than a transistor including amorphous silicon. In addition, the OS transistor has an extremely low leakage current between a source and a drain in an off state (hereinafter also referred to as off-state current), and electric charge accumulated in a capacitor that is connected in series to the transistor can be retained for a long period. Furthermore, the power consumption of the display panel can be reduced with the OS transistor.

To increase the emission luminance of the light-emitting device included in the pixel circuit, the amount of current fed through the light-emitting device needs to be increased. To increase the current amount, the source–drain voltage of a driving transistor included in the pixel circuit needs to be increased. An OS transistor has a higher breakdown voltage between a source and a drain than a Si transistor; hence, high voltage can be applied between the source and the drain of the OS transistor. Therefore, when an OS transistor is used as the driving transistor in the pixel circuit, the amount of current flowing through the light-emitting device can be increased, so that the emission luminance of the light-emitting device can be increased.

Assuming that the transistor operates in a saturation region, a change in the amount of current between the source and the drain, with respect to a fluctuation in the gate–source voltage, in the OS transistor is smaller than that in the Si transistor. Accordingly, when an OS transistor is used as the driving transistor included in the pixel circuit, a current flowing between the source and the drain can be set minutely in accordance with a change in gate–source voltage; hence, the amount of current flowing through the light-emitting device can be controlled. Consequently, the number of gray levels expressed by the pixel circuit can be increased.

Regarding saturation characteristics of a current flowing when transistors operate in a saturation region, even in the case where the source–drain voltage of an OS transistor increases gradually, a more stable current (saturation current) can be fed through the OS transistor than through a Si transistor. Thus, by using an OS transistor as the driving transistor, a stable current can be fed through light-emitting devices even when the current-voltage characteristics of the light-emitting devices vary, for example. In other words, when the OS transistor operates in the saturation region, the source–drain current hardly changes with an increase in the source–drain voltage; hence, the emission luminance of the light-emitting device can be stable.

### Display panel 200E

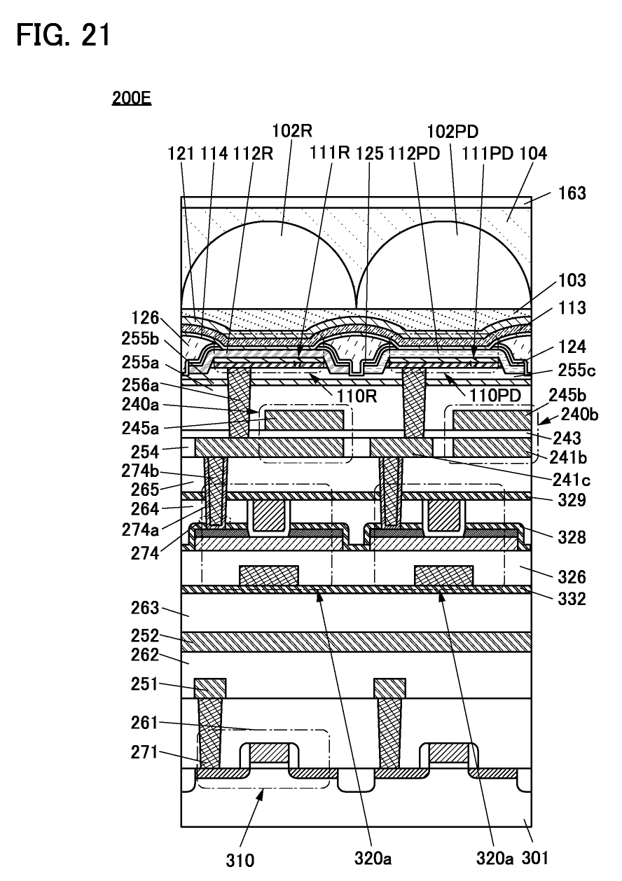

The display panel 200E illustrated in FIG. 21 has a structure in which the transistor 310 having a channel formed in the substrate 301 and the transistor 320 (transistors 320a and 320b) each including a metal oxide in a semiconductor layer where a channel is formed are stacked.

The insulating layer 261 is provided to cover the transistor 310, and a conductive layer 251 is provided over the insulating layer 261. An insulating layer 262 is provided to cover the conductive layer 251, and a conductive layer 252 is provided over the insulating layer 262. The conductive layer 251 and the conductive layer 252 each function as a wiring. An insulating layer 263 and the insulating layer 332 are provided to cover the conductive layer 252, and the transistor 320 is provided over the insulating layer 332.

The transistor 320 can be used as a transistor included in the pixel circuit. The transistor 310 can be used as a transistor included in the pixel circuit or a transistor included in a driver circuit for driving the pixel circuit (a gate line driver circuit or a source line driver circuit). The transistor 310 can also be used as transistors included in a variety of circuits such as an arithmetic circuit and a memory circuit.

With such a structure, not only the pixel circuit but also the driver circuit and the like can be formed directly under the light-emitting elements and the light-receiving element; thus, the display panel can be downsized as compared with the case where a driver circuit is provided around a display region.

### Display panel 200F

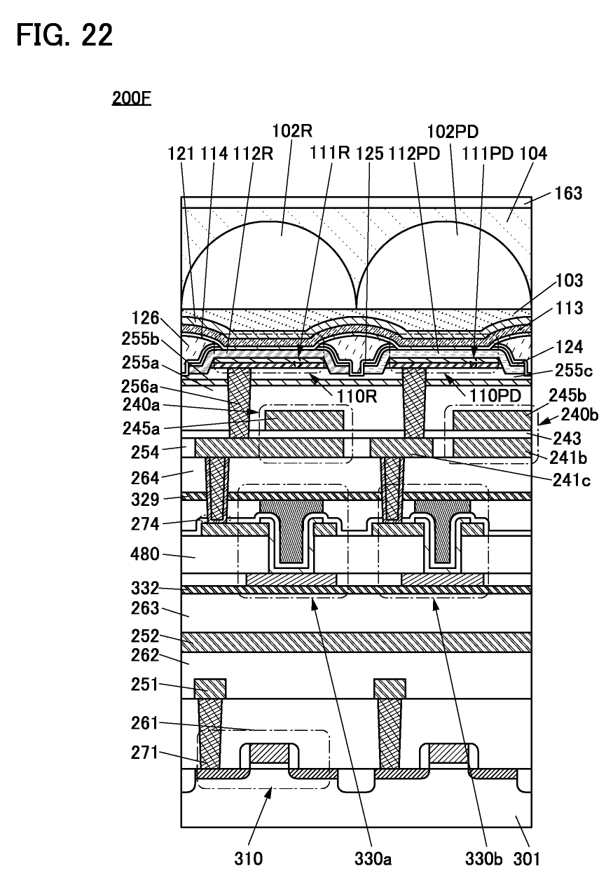

A display panel 200F illustrated in FIG. 22 has a structure in which the transistor 320 in the display panel 200E illustrated in FIG. 21 is replaced with a transistor 330 (a vertical transistor: a transistor 330a and a transistor 330b). Note that the structure in which the transistor 320 is replaced with the transistor 330 can also be employed for the display panel 200D illustrated in FIG. 20.

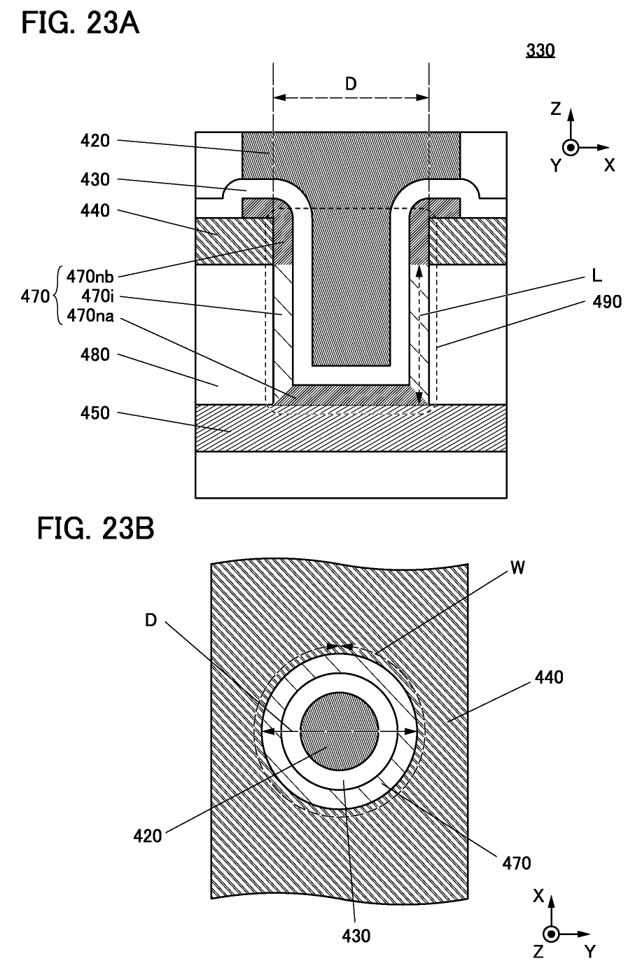

FIG. 23A is a cross-sectional view of the transistor 330 along the X–Z plane. FIG. 23B is a cross-sectional view along the X–Y plane including a wiring 440.

The transistor 330 includes an oxide semiconductor 470, an insulator 430, and a conductor 420. The oxide semiconductor 470 functions as a semiconductor layer, the insulator 430 functions as a gate insulator, and the conductor 420 functions as a gate electrode. A wiring 450 includes a region functioning as one of a source electrode and a drain electrode of the transistor 330. The wiring 440 includes a region functioning as the other of the source electrode and the drain electrode of the transistor 330.

An opening portion 490 penetrating the wiring 440 and an insulator 480 and reaching the wiring 450 is provided. The opening portion 490 has a pillar shape with a substantially circular top surface. This structure enables miniaturization or high integration of the memory cell. Note that the side surface of the opening portion 490 is preferably perpendicular to the top surface of the wiring 450.

At least part of the oxide semiconductor 470 is provided in the opening portion 490. Note that the oxide semiconductor 470 includes a region in contact with the top surface of the wiring 450, a region in contact with the side surface of the wiring 440, and a region in contact with the side surface of the insulator 480 in the opening portion 490.

The insulator 430 is provided so as to at least partly cover the opening portion 490. The conductor 420 is provided so that at least part of the conductor 420 is positioned in the opening portion 490. The conductor 420 is preferably provided so as to be embedded in the opening portion 490, and the top-view shape of the conductor 420 is preferably substantially circular for a higher integration degree.

As illustrated in FIG. 23A, the oxide semiconductor 470 includes a region 470i and regions 470na and 470nb provided with the region 470i positioned therebetween.

The region 470na is a region of the oxide semiconductor 470 that is in contact with the wiring 450. At least part of the region 470na functions as one of a source region and a drain region of the transistor 330. The region 470nb is a region of the oxide semiconductor 470 that is in contact with the wiring 440. At least part of the region 470nb functions as the other of the source region and the drain region of the transistor 330. As illustrated in FIG. 23B, the wiring 440 is in contact with all the perimeter of the oxide semiconductor 470. Thus, the other of the source region and the drain region of the transistor 330 can be formed along all the perimeter of a portion formed in the same layer as the wiring 440 in the oxide semiconductor 470.

The region 470i is a region interposed between the regions 470na and 470nb in the oxide semiconductor 470. At least part of the region 470i functions as the channel formation region of the transistor 330. That is, the channel formation region of the transistor 330 is formed in part of the oxide semiconductor 470 that is positioned in a region between the wirings 450 and 440. It can be said that the channel formation region of the transistor 330 is positioned in a region in contact with the insulator 480 or a region in the vicinity thereof in the oxide semiconductor 470.

The channel length of the transistor 330 is a distance between the source region and the drain region. That is, the channel length of the transistor 330 is determined by the thickness of the insulator 480 over the wiring 450. In FIG. 23A, a channel length L of the transistor 330 is indicated by a dashed double-headed arrow. The channel length L is a distance between an end portion of a region where the oxide semiconductor 470 and the wiring 450 are in contact with each other and an end portion of a region where the oxide semiconductor 470 and the wiring 440 are in contact with each other in a cross-sectional view. That is, the channel length L corresponds to the length of the side surface of the insulator 480 on the opening portion 490 side in the cross-sectional view.

The channel length of a planar transistor is limited by the light exposure limit of photolithography, and further miniaturization is difficult. By contrast, in one embodiment of the present invention, the channel length can be determined by the thickness of the insulator 480. Thus, the transistor 330 can have an extremely small channel length less than or equal to the light exposure limit of photolithography (e.g., less than or equal to 60 nm, less than or equal to 50 nm, less than or equal to 40 nm, less than or equal to 30 nm, less than or equal to 20 nm, or less than or equal to 10 nm, and greater than or equal to 1 nm, or greater than or equal to 5 nm). Accordingly, the transistor 330 can have a high on-state current.

In addition, as described above, the channel formation region, the source region, and the drain region can be formed in the opening portion 490. Thus, the area occupied by the transistor 330 can be reduced as compared with a planar transistor in which the channel formation region, the source region, and the drain region are provided separately on the X–Y plane. Accordingly, the pixel density can be increased.

Such a s transistor including the channel formation region along the side surface of the insulator 480 in the opening portion 490 is referred to as a vertical transistor.

Furthermore, in the X–Y plane including the channel formation region of the oxide semiconductor 470, as illustrated in FIG. 23B, the oxide semiconductor 470, the insulator 430, and the conductor 420 are provided concentrically. Therefore, the side surface of the conductor 420 provided at the center faces the side surface of the oxide semiconductor 470 with the insulator 430 therebetween. That is, in the top view, all the perimeter of the oxide semiconductor 470 serves as the channel formation region. In this case, for example, the channel width of the transistor 330 is determined by the length of the perimeter of the oxide semiconductor 470. In other words, the channel width of the transistor 330 is determined by the maximum width of the opening portion 490 (the diameter in the case where the opening portion 490 is circular in the top view). In FIGS. 23A and 23B, a maximum width D of the opening portion 490 is indicated by a dashed double-dotted double-headed arrow. In FIG. 23B, a channel width W of the transistor 330 is indicated by a dashed-dotted double-headed arrow. By increasing the maximum width D of the opening portion 490, the channel width per unit area can be increased and the on-state current can be increased.

In the case where the opening portion 490 is formed by a photolithography method, the maximum width D of the opening portion 490 is limited by the light exposure limit of photolithography. In addition, the maximum width D of the opening portion 490 is determined by the film thicknesses of the oxide semiconductor 470, the insulator 430, and the conductor 420 provided in the opening portion 490. The maximum width D of the opening portion 490 is preferably, for example, greater than or equal to 5 nm, greater than or equal to 10 nm, or greater than or equal to 20 nm and less than or equal to 100 nm, less than or equal to 60 nm, less than or equal to 50 nm, less than or equal to 40 nm, or less than or equal to 30 nm. In the case where the opening portion 490 is circular in the top view, the maximum width D of the opening portion 490 corresponds to the diameter of the opening portion 490, and the channel width W can be "D × π".

In the memory device of one embodiment of the present invention, the channel length L of the transistor 330 is preferably shorter than at least the channel width W of the transistor 330. The channel length L of the transistor 330 of one embodiment of the present invention is greater than or equal to 0.1 times and less than or equal to 0.99 times, preferably greater than or equal to 0.5 times and less than or equal to 0.8 times the channel width W of the transistor 330. This structure enables a transistor with favorable electrical characteristics and high reliability.

In the case where the opening portion 490 is formed to be substantially circular in the top view, the oxide semiconductor 470, the insulator 430, and the conductor 420 are provided concentrically. This makes the distance between the conductor 420 and the oxide semiconductor 470 substantially uniform, so that a gate electric field can be substantially uniformly applied to the oxide semiconductor 470.

It is preferable that the channel formation region of the transistor including an oxide semiconductor in the semiconductor layer contain less oxygen vacancies or have a lower concentration of impurities such as hydrogen, nitrogen, or a metal element than the source region and the drain region. For example, the concentration of aluminum in the channel formation region of the oxide semiconductor is preferably lower than or equal to 1 × 10²² atoms/cm³, further preferably lower than or equal to 1 × 10²¹ atoms/cm³, still further preferably lower than or equal to 1 × 10²⁰ atoms/cm³, yet further preferably lower than or equal to 5 × 10¹⁹ atoms/cm³, yet still further preferably lower than or equal to 1 × 10¹⁹ atoms/cm³, yet still further preferably lower than or equal to 5 × 10¹⁸ atoms/cm³, yet still further preferably lower than or equal to 1 × 10¹⁸ atoms/cm³.

In some cases, hydrogen in the vicinity of an oxygen vacancy forms a defect that is an oxygen vacancy into which hydrogen enters (hereinafter sometimes referred to as V_{O}H), which generates an electron serving as a carrier. Thus, it is preferable that the amount of V_{O}H be also reduced in the channel formation region. Thus, the channel formation region of the transistor is a high-resistance region having a low carrier concentration. Accordingly, the channel formation region of the transistor can be regarded as an i-type (intrinsic) or substantially i-type region.

The source region and the drain region of the transistor including an oxide semiconductor in the semiconductor layer are regions which have lower resistances than the channel formation region by having increased carrier concentrations because of including more oxygen vacancies, including a larger amount of V_{O}H, or having higher concentrations of impurities such as hydrogen, nitrogen, or a metal element. In other words, the source region and the drain region of the transistor are n-type regions having higher carrier concentrations and lower resistances than the channel formation region.

Although the opening portion 490 is provided so that the side surface of the opening portion 490 is perpendicular to the top surface of the wiring 450 in FIG. 23A and the like, the present invention is not limited thereto. For example, the side surface of the opening portion 490 may have a tapered shape.

At least part of this embodiment can be implemented in combination with any of the other embodiments and an example described in this specification as appropriate.

### Embodiment 3

In this embodiment, an indium oxide film that can be used for the semiconductor layer of the transistor included in the display panel of one embodiment of the present invention will be described.

In this specification and the like, indium oxide including at least a crystal part or a crystal region in a film is referred to as crystal IO or crystalline IO. Examples of crystal IO or crystalline IO include single crystal indium oxide, polycrystal indium oxide, and microcrystal indium oxide.

Indium oxide is a semiconductor material having physical properties completely different from those of an oxide semiconductor such as In-Ga-Zn oxide (hereinafter, also referred to as IGZO) or zinc oxide.

The dependence of the Hall mobility on the carrier concentration of indium oxide, silicon, and IGZO will be described.

IGZO has a tendency in which the Hall mobility is higher as the carrier concentration is higher. By contrast, single crystal indium oxide has a tendency in which the Hall mobility is higher as the carrier concentration is lower. This tendency is similar to that of silicon; as the concentration of a dopant (impurity) in a material is lower, impurity scattering is inhibited more and thus the Hall mobility is higher. That is, single crystal indium oxide having higher purity and being more intrinsic has higher Hall mobility. Consequently, the physical properties of single crystal indium oxide are different from those of IGZO and similar to those of silicon. Note that the tendency of non-single-crystal (e.g., polycrystal) indium oxide is sometimes different from that of single crystal indium oxide.

A carrier concentration range suitable for a channel formation region of a transistor is a range including a carrier concentration of 1 × 10¹⁵ cm⁻ ³, e.g., a range with a carrier concentration higher than or equal to 1 × 10¹⁴ cm⁻ ³ and lower than or equal to 1 × 10¹⁸ cm⁻ ³. The adequately lowered carrier concentration will increase the Hall mobility to approximately 270 cm²/(V⋅s).

Indium oxide can include an element that reduces the carrier concentration. Examples of the element that reduces the carrier concentration include magnesium, calcium, zinc, cadmium, and copper. When indium is replaced with any of these elements, the carrier concentration can be reduced. Other examples include nitrogen, phosphorus, arsenic, and antimony. When oxygen is replaced with any of these elements, the carrier concentration can be reduced.

The element that reduces the carrier concentration can be regarded as an element that imparts p-type conductivity. When the element that reduces the carrier concentration is added to an indium oxide film, the indium oxide film is easily i-type or substantially i-type in some cases. In that case, the concentration of the element that reduces the carrier concentration in the indium oxide film is preferably higher than or equal to 1 × 10¹⁶ atoms/cm³ and lower than or equal to 8 × 10²⁰ atoms/cm³, more preferably higher than or equal to 1 × 10¹⁶ atoms/cm³ and lower than or equal to 4 × 10²⁰ atoms/cm³, further preferably higher than or equal to 1 × 10¹⁶ atoms/cm³ and lower than or equal to 8 × 10¹⁹ atoms/cm³, still more preferably higher than or equal to 1 × 10¹⁶ atoms/cm³ and lower than or equal to 5 × 10¹⁹ atoms/cm³. With such a concentration, the carrier concentration can be reduced while the crystal structure of indium oxide is maintained.

By contrast, an increase in carrier concentration can reduce electrical resistance. A carrier concentration range suitable for a source region and a drain region of a transistor, a resistor, or a transparent conductive film is, for example, a range including a carrier concentration of 1 × 10²⁰ cm⁻ ³, e.g., a range with a carrier concentration higher than or equal to 1 × 10¹⁹ cm⁻ ³ and lower than or equal to 1 × 10²² cm⁻ ³. The adequately increased carrier concentration will decrease the resistivity to 1 × 10⁻ ⁴ Ω⋅cm or lower.

Indium oxide can include an element that increases the carrier concentration. For example, the same element as a source electrode and a drain electrode of a transistor is preferably included. Examples of the element that increases the carrier concentration include titanium, zirconium, hafnium, tantalum, tungsten, molybdenum, tin, silicon, and boron. It is particularly preferable that an oxide of the element have conductivity or semiconductor properties.

The element that increases the carrier concentration can also be regarded to as an element that imparts n-type conductivity. The concentration of the element that increases the carrier concentration in the indium oxide film is preferably higher than or equal to 1 × 10¹⁶ atoms/cm³ and lower than or equal to 8 × 10²⁰ atoms/cm³, more preferably higher than or equal to 1 × 10¹⁶ atoms/cm³ and lower than or equal to 4 × 10²⁰ atoms/cm³, further preferably higher than or equal to 1 × 10¹⁶ atoms/cm³ and lower than or equal to 8 × 10¹⁹ atoms/cm³, still more preferably higher than or equal to 1 × 10¹⁶ atoms/cm³ and lower than or equal to 5 × 10¹⁹ atoms/cm³. With such a concentration, the carrier concentration can be increased while the crystal structure of indium oxide is maintained. Note that antimony sometimes functions as an element that increases the carrier concentration.

As described above, indium oxide is an oxide whose valence electron can be controlled; therefore, the threshold voltage of a transistor using a semiconductor layer containing indium oxide can be controlled. In addition, the region with a low carrier concentration and the region with a high carrier concentration of indium oxide can be used as a channel formation region and source and drain regions, respectively, of a transistor. Thus, the indium oxide enables formation of what is called an n-i-n junction (junction between an n-type region, an i-type region, and an n-type region). Incidentally, the valence electron control by a transistor with silicon is generally known, whereas the valence electron control in a transistor containing indium oxide is a novel technical idea that cannot be conceived usually. With the use of this technical idea, a transistor that has high mobility, has a low off-state current, can be normally off, and has high reliability can be achieved.

An indium oxide film preferably has crystallinity. In particular, the indium oxide film is preferably a polycrystal film, further preferably a single crystal film. A single crystal film does not have a crystal grain boundary (also referred to as a grain boundary). The use of a single crystal film can inhibit carrier scattering or the like at the crystal grain boundary, thereby achieving a transistor having high field-effect mobility. In addition, the use of a single crystal film produces an excellent effect of reducing a variation in transistor characteristics caused by the crystal grain boundary.

A polycrystal film is preferable because it can reduce carrier scattering as compared with a microcrystal film or an amorphous film and enables a transistor to have high field-effect mobility. In the case of using a polycrystal film, it is preferable to use a film that has as large a crystal grain size as possible and few crystal grain boundaries. In the case where the crystal grain boundary is neither included nor observed in a channel formation region of a transistor including a polycrystal film, the channel formation region is positioned in a single crystal region included in the polycrystal film and thus the transistor can be regarded as a transistor including a single crystal film.

The crystallinity of indium oxide can be analyzed with an X-ray diffraction (XRD) pattern, a transmission electron microscope (TEM) image, or an electron diffraction (ED) pattern, for example. Alternatively, two or more of these methods may be combined for the analysis.

In this specification and the like, a semiconductor layer where no crystal grain boundary is observed in a channel formation region, a semiconductor layer where a channel formation region is included in one crystal grain, or a semiconductor layer where the directions of crystal axes of at least two regions in a channel formation region are the same can be regarded as a single crystal film.

A channel formation region refers to a region of a semiconductor layer that overlaps with (or faces) a gate electrode with a gate insulating layer therebetween and is positioned between a region in contact with a source electrode and a region in contact with a drain electrode. A crystal grain, a crystal grain boundary, a crystal axis, a crystal orientation, or the like in a channel formation region can be confirmed in observation of a cross section including a semiconductor layer, a source electrode, and a drain electrode.

Impurities in the indium oxide film can function as a carrier scattering source and thus can cause a reduction in field-effect mobility and inhibit crystal growth. Examples of the impurities for the indium oxide film include boron and silicon. The concentrations of the impurities in the indium oxide film in the channel formation region are preferably as low as possible. For example, the concentrations of the impurity elements are each lower than or equal to 0.1 %, preferably lower than or equal to 0.01 % (100 ppm). Note that carbon, hydrogen, and the like are elements that would be contained in a film formation gas or a precursor in film formation, and the amounts of these elements remaining in the indium oxide film might be larger than those of the impurities.

The indium oxide film may contain an element that can form a trivalent cation like indium as long as the cubic crystal structure (bixbyite structure) is retained. Examples of the element include Group 13 elements such as gallium and aluminum and Group 3 elements in the periodic table. These elements have a higher bonding strength with oxygen than indium, and therefore, addition of any of these elements to an indium oxide film can inhibit formation of oxygen vacancies (V_{O}) in the indium oxide film. Since these elements exist mainly as trivalent cations in oxides, the carrier concentration in the indium oxide film can be kept low. This enables the threshold voltage of a transistor to shift in the positive direction and the transistor to exhibit normally-off characteristics. Consequently, the transistor can have favorable electrical characteristics.

The concentration of the above element in the indium oxide film is preferably higher than or equal to 1 × 10¹⁶ atoms/cm³ and lower than or equal to 8 × 10²⁰ atoms/cm³, more preferably higher than or equal to 1 × 10¹⁶ atoms/cm³ and lower than or equal to 4 × 10²⁰ atoms/cm³, further preferably higher than or equal to 1 × 10¹⁶ atoms/cm³ and lower than or equal to 8 × 10¹⁹ atoms/cm³, still more preferably higher than or equal to 1 × 10¹⁶ atoms/cm³ and lower than or equal to 5 × 10¹⁹ atoms/cm³.

The element that imparts n-type conductivity, the element that imparts p-type conductivity, and the element that can form a trivalent cation described above can each be added to an indium oxide film by an ion doping method, an ion implantation method, a thermal diffusion method, or the like. Alternatively, the element that imparts n-type conductivity, the element that imparts p-type conductivity, and the element that can form a trivalent cation described above can each be added to a sputtering target, a deposition gas, a precursor for ALD deposition, or the like at the time of depositing an indium oxide film. Alternatively, it is possible to perform autodoping with the element that imparts n-type conductivity, the element that imparts p-type conductivity, and the element that can form a trivalent cation described above from a chamber in depositing the indium oxide film. Note that autodoping refers to addition of an element contained in a member of a chamber for deposition, for example, into the film at the time of depositing the indium oxide film.

A transistor including the above indium oxide film can have a field-effect mobility higher than or equal to 50 cm²/(V⋅s), preferably higher than or equal to 100 cm²/(V⋅s), further preferably higher than or equal to 150 cm²/(V⋅s), still further preferably higher than or equal to 200 cm²/(V⋅s), yet still further preferably higher than or equal to 250 cm²/(V⋅s).

One feature of an indium oxide film is to have a higher property of transmitting (diffusing) oxygen than an IGZO film. For example, oxygen diffusing in an indium oxide film is transmitted through the indium oxide film and released as an oxygen molecule. When reacting with hydrogen contained in the film, oxygen is released as a water molecule in some cases. In the case where the film includes oxygen vacancies, the oxygen vacancies are filled with diffusing oxygen atoms. Since oxygen easily diffuses in the indium oxide film, oxygen vacancies in the indium oxide film are filled with oxygen more easily than those in an IGZO film.

As described above, the oxygen vacancies in the indium oxide film are reduced more easily than those in the IGZO film; thus, a transistor including such an indium oxide film can have extremely high reliability.

Hydrogen diffuses in the indium oxide film. Hydrogen diffusing into the indium oxide film from the outside is transmitted through the indium oxide film and is released as a hydrogen molecule. When reacting with oxygen contained in the film, hydrogen is released as a water molecule.

Indium oxide has features of a small effective mass of electrons and a large effective mass of holes. In addition, the effective mass of electrons in indium oxide hardly depends on the crystal orientation. Thus, a transistor containing indium oxide having crystallinity can have high field-effect mobility and high frequency characteristics (also referred to as f characteristics). A large effective mass of holes allows a transistor to have an extremely low off-state current. For example, the off-state current per micrometer of channel width of a vertical transistor including an indium oxide film can be lower than or equal to 1 fA (1 × 10⁻ ¹⁵ A) or lower than or equal to 1 aA (1 × 10⁻ ¹⁸ A) at 125 °C, and can be lower than or equal to 1 aA (1 × 10⁻ ¹⁸ A) or lower than or equal to 1 zA (1 × 10⁻ ²¹ A) at room temperature (25 °C). Since indium oxide has a smaller effective mass of electrons and a larger effective mass of holes than silicon, a transistor containing indium oxide can have higher field-effect mobility and lower off-state current than a Si transistor.

A seed layer is preferably provided in contact with at least part of the indium oxide film having crystallinity. A material of the seed layer is preferably selected such that the difference in a lattice constant (also referred to as lattice mismatch) between the crystal included in indium oxide and the crystal included in the material is small. In that case, the crystallinity of the indium oxide film can be improved. As a layer in contact with at least part of the indium oxide film having crystallinity, a substrate (e.g., a single crystal substrate) may be used.

One of methods for evaluating the degree of a lattice mismatch is a method using a value of a lattice mismatch degree described below. A lattice mismatch degree Δa [%] of a crystal included in a film to be formed (here, the indium oxide film) with respect to the crystal included in the seed layer is calculated by the formula: Δa = ((L₁ - L₂)/L₂) × 100. Here, L₁ is the lattice constant or the length of the unit lattice vector of the crystal included in the film to be formed, and L₂ is the lattice constant or the length of the unit lattice vector of the crystal included in the seed layer.

The absolute value of the lattice mismatch degree Δa between the seed layer and the indium oxide film is preferably as small as possible, most preferably 0. For example, Δa can be greater than or equal to -5 % and less than or equal to 5 %, preferably greater than or equal to -4 % and less than or equal to 4 %, further preferably greater than or equal to -3 % and less than or equal to 3 %, still further preferably greater than or equal to -2 % and less than or equal to 2 %.

An indium oxide crystal has a cubic crystal structure (a bixbyite structure). For example, an yttria-stabilized zirconia (YSZ) crystal can have a cubic crystal structure (a fluorite crystal structure). The lattice mismatch degree of an indium oxide crystal with respect to an YSZ crystal having the cubic crystal structure is within the range of -2 % to 2 %, which enables epitaxial growth of a single crystal film of indium oxide over the YSZ substrate.

The crystal structures of the seed layer and the indium oxide film do not necessarily have the same crystal system or crystal orientation in some cases. For example, a film including a crystal with a hexagonal crystal structure or a trigonal crystal structure can be provided below an indium oxide film including a crystal with a cubic crystal structure. For example, when the crystal orientation of a seed layer surface is set to [001] and the crystal orientation of a bottom surface of the indium oxide film is set to [111], the necessary condition for crystal orientation in epitaxial growth can be satisfied. Examples of a hexagonal or trigonal crystal structure include a wurtzite structure, a YbFe₂O₄-type structure, a Yb₂Fe₃O₇-type structure, and variations of these structures. An example of a crystal having a YbFe₂O₄-type structure or a Yb₂Fe₃O₇-type structure is IGZO.

At least part of this embodiment can be implemented as appropriate in combination with any of the other embodiments described in this specification.

This application is based on Japanese Patent Application Serial No. 2025-073361 filed with Japan Patent Office on Apr. 25, 2025, the entire contents of which are hereby incorporated by reference.

What is claimed is:

1. An electronic device comprising:
a display panel;
a first optical system;
a second optical system; and
a position adjustment mechanism,
wherein the display panel comprises a pixel comprising a first light-emitting element, a second light-emitting element, and a light-receiving element,
wherein the first light-emitting element is configured to emit visible light,
wherein the second light-emitting element is configured to emit infrared light,
wherein the light-receiving element is configured to perform photoelectric conversion of the infrared light,
wherein the first optical system faces a display surface of the display panel,
wherein the first optical system comprises a half mirror and a reflective polarizing plate,
wherein the half mirror is configured to partially transmit and partially reflect the visible light and to transmit the infrared light,
wherein the reflective polarizing plate is configured to transmit or reflect the visible light in accordance with a polarization state of the visible light and to transmit the infrared light regardless of a polarization state of the infrared light,
wherein the position adjustment mechanism is configured to move the second optical system to a first position or a second position,
wherein the visible light emitted from the pixel forms an image on an object through the first optical system when the second optical system is at the first position, and
wherein the infrared light emitted from the pixel forms an image on the object through the second optical system and the first optical system and infrared light reflected by the object forms an image on the pixel through the first optical system and the second optical system when the second optical system is at the second position.

2. The electronic device according to claim 1,
wherein in the first optical system, a linear polarizing plate, a first retardation plate, the half mirror, a second retardation plate, and the reflective polarizing plate are arranged in one direction in this order from a display panel side.

3. The electronic device according to claim 1,
wherein the second optical system has a positive power.

4. The electronic device according to claim 1,
wherein the first position is out of an optical path of light that is emitted from the pixel and reaches the first optical system, and
wherein the second position is in the optical path of the light that is emitted from the pixel and reaches the first optical system.

5. The electronic device according to claim 1,
wherein the first light-emitting element and the second light-emitting element are organic EL elements, and the light-receiving element is a photoelectric conversion element comprising an organic layer used for a photoelectric conversion layer.

6. The electronic device according to claim 1,
wherein the position adjustment mechanism comprises a stepper motor.

7. The electronic device according to claim 1,
wherein the pixel comprises a transistor connected to the first light-emitting element, the second light-emitting element, and the light-receiving element, and the transistor comprises a metal oxide in a channel formation region.

8. The electronic device according to claim 7,
wherein the metal oxide comprises indium and oxygen.

9. A method of operating an electronic device worn in front of an eye, the electronic device comprising a first optical unit corresponding to one of left and right eyes and a second optical unit corresponding to the other of the left and right eyes,
the method comprising:
switching a first mode and a second mode,
wherein, in the first mode, visible light is emitted from a pixel of a display panel included in each of the first optical unit and the second optical unit, and the visible light forms an image on an object through a first optical system included in each of the first optical unit and the second optical unit, and
wherein, in the second mode, in one of the first optical unit and the second optical unit, display with the visible light is performed on the display panel to adjust a thickness of a crystalline
lens of each of the eyes, and in the other of the first optical unit and the second optical unit, a second optical system is inserted between the display panel and the first optical system, infrared light is emitted from the pixel of the display panel, the infrared light forms an image on the object through the second optical system and the first optical system, and infrared light reflected by the object forms an image on the pixel through the first optical system and the second optical system.
